(19) United States
(12) Patent Application Publication
(10) Pub. No.: US 2025/0321216 A1
(43) Pub. Date: Oct. 16, 2025

(54) Systems and Methods for Elemental Soil Content Determination Utilizing Inelastic Neutron Scattering and Accounting for Moisture Content and Ambient Temperature

(71) Applicants: The United States of America, as represented by the Secretary of Agriculture, Washington, DC (US); Carbon Asset Solutions (USA), Inc., Wetumpka, AL (US)

(72) Inventors: Henry Allen Torbert, III, Auburn, AL (US); Galina N. Yakubova, Auburn, AL (US); Aleksandr G. Kavetskiy, Auburn, AL (US); Stephen A. Prior, Auburn, AL (US); Nikolay Sargsyan, Sippy Downs (AU)

(73) Assignees: The United States of America, as represented by the Secretary of Agriculture, Washington, DC (US); Carbon Asset Solutions (USA), Inc., Wetumpka, AL (US)

(21) Appl. No.: 18/634,535

(22) Filed: Apr. 12, 2024

(51) Int. Cl. G01N 33/24; G01N 23/20008; G01N 23/20066

(52) U.S. Cl. CPC G01N 33/246; G01N 23/20008; G01N 23/20066; G01N 33/245; G01N 2223/1063; G01N 2223/1066; G01N 2223/303; G01N 2223/305; G01N 2223/3103; G01N 2223/3106; G01N 2223/402; G01N 2223/613; G01N 2223/616

(57) ABSTRACT

Systems and methods for measuring the content of an element in a soil comprises: a neutron source for irradiating the soil with neutrons; a detector assembly configured to detect an INS gamma spectrum of the soil; an instrument for measuring a moisture content of the soil; and a processor in communication with the detector assembly, the processor configured to: apply a moisture calibration coefficient to calculate a net peak area of a characteristic peak of the element in the INS gamma spectrum, the moisture calibration coefficient calculated to account for the moderation of fast neutrons by hydrogen atoms of water present in the irradiated soil at the location under analysis; and generate a concentration of the element in the soil. The instrument for measuring a moisture content of the soil may comprise the detector assembly. Systems and methods incorporating a temperature-controlled housing for the gamma detector assembly are also provided.

### TECHNICAL FIELD

The present disclosure relates to systems and methods for mapping a distribution of at least one compound within soil.

### BACKGROUND

Elemental content analysis of the soil of a given geographic area may reveal whether the soil is adaptable to particular uses, such as agricultural, recreational, and so on.

Other uses of soil content analysis include obtaining documentation required for claiming carbon credits and analyzing soil conditions and yield potential for precision agriculture and other management practices.

Other uses of soil content analysis include obtaining documentation required for claiming carbon credits and analyzing the availability of nutrients or the need for nutrient introduction to evaluate present and projected yields and potential profitability of fertilization.

Soil analysis may begin with soil sample collection, such that only a tiny portion of a field is analyzed in the laboratory. For example, one common method of soil elemental content analysis is composite sampling, where several subsamples of the soil are collected from randomly selected locations in the field. The subsamples are then mixed and the mixture is analyzed for elemental content. In some instances, a quantity of a given element revealed to be contained within the mixture may be treated as an average quantity of that element within the entire area of the field being analyzed.

While an actual number of subsamples may vary slightly based on field size and uniformity, a number of subsamples usually does not exceed 20 and, at times, amounts to less than 0.01% of the acreage being analyzed. Moreover, most soil testing and analysis systems are not readily adaptable to test more than a few samples and, at best, provide a high-level approximation of a true elemental content of the soil of the field. As it is desirable to obtain accurate analysis of elemental content of the soil and to determine the variability of the elemental content across a geographic area, a methodology yielding more detailed and accurate elemental content information for a given field area is needed.

As described in the Applicant's U.S. patent application Ser. No. 16/706,013 (issued U.S. Pat. No. 11,397,277) and U.S. patent application Ser. No. 17/841,952, systems and methods have been developed for analyzing the content of at least one element in the soil of a field by detecting and analyzing gamma spectra obtained from soil samples distributed across a field, using a mobile cart. The system mounted to the mobile cart may include a neutron generator device, a plurality of gamma detectors (for example, sodium iodine gamma detectors) for scanning at least a portion of the field, and a computing system for storing and analyzing the results of the scan and generating a map indicating the elemental content of at least a portion of the field. The mobile cart may be configured to travel over a substantial portion of the field to perform the scan of the soil and acquire the gamma spectra for analysis.

The content of the elements C, Si, H and/or K, amongst others, may be calculated using the acquired spectra captured by the gamma detectors.

However, the Applicant has found that different environmental factors, including temperature fluctuations and moisture content in the soil, may affect the accuracy of the resulting soil content analysis obtained from the acquired gamma spectra. Thus, it is desirable to control for these environmental factors to obtain a more accurate analysis of the elemental soil content utilizing neutron-induced gamma analysis.

In addition to analyzing the soil of a geographic area to determine the elemental content of the soil (such as, the percentage of Carbon and Silicon present in the soil), it may also be useful to determine the moisture content of the soil. For example, knowledge about soil moisture content may have important implications for selecting appropriate tillage practice and irrigation management. The quantity of water in soil may be expressed by one of two units: either the gravimetric water content (ie: the mass of the water per unit of mass of dry soil), or the volumetric water content (ie: the volume of water per unit of volume of soil).

A commonly accepted standard method of measuring the water content of soil is via the gravimetric method, whereby a subsample of a fresh, sieved composite sample of the soil, or a subsample of a fresh soil core, is weighed. Then the subsample is oven dried until no further mass loss occurs and then re-weighed. The difference in mass is attributed to water that evaporated from the subsample, and the moisture content is expressed as mass of water per mass of dry soil. Another method involves neutron ray moderation, whereby neutrons are moderated to thermal energy by the hydrogen nuclei present in water molecules; or in other words, measuring the thermal flux of a neutron ray focused on the soil sample to thereby calculate the water content of the soil. Yet another method for measuring water content in soil involves time domain reflectometry (TDR), which is performed with a device that measures permittivity, or in other words the dielectric number, of the soil, and then performs calculations to convert the permittivity reading into a volumetric water content. Using the known value of soil density, as would be known to a person skilled in the art, the volumetric water content of the soil may be recalculated to determine the gravimetric water content in the soil sample under analysis.

A disadvantage of the above-mentioned methods for determining soil moisture content is that each method involves measuring a small volume of soil and distributing the resulting moisture content value over a large geographic area. Because soil moisture may fluctuate greatly across a geographic area, the typical method of measuring only a small number of samples by the methods described above across a larger geographic area, and then generalizing those results across the entire geographic area to be measured, may result in an inaccurate measurement of the soil moisture content at any particular point across that geographic area. Additionally, the application of each method described above requires taking the samples from the field, or in the case of TDR, inserting electrodes into the soil at selected points across the geographic area; each method is a time-consuming and labor intensive process.

### SUMMARY

In one aspect of the present disclosure, a method and system for determining the content of at least one element in a soil over a geographic area is provided. An example system for developing a detailed and accurate map of the elemental content of soil of a given field or other geographical area may include a neutron generator device and a plurality of gamma detector assemblies (e.g., sodium iodine gamma detectors, which assembly may each comprise a sodium iodine crystal operatively coupled to a photomultiplier tube (PMT)). The neutron generator device and the plurality of gamma detector assemblies are configured for scanning at least a portion of the field and a computing system for storing and analyzing the results of the scan and generating a map indicative of elemental content of the portion of the field. The system may be a mobile system and may be configured to travel over a substantial portion of the field to perform the scan of the soil. According to some embodiments of the present disclosure, the elemental (C, Si, O, H, K, CI, and others) content in soil may be calculated using the measured spectra captured by the gamma detectors.

The example system may be further configured to communicate with a global positioning system (GPS) device to capture geographic location of the soil during the scanning process. In one example, the elemental content data identified during the scan may be combined (or associated) with geographic coordinates provided by the GPS device. Additionally, or alternatively, based on the elemental content determined from the scan and the associated geographic coordinates, the example system may be configured to generate an element distribution map suitable for agricultural or other purposes.

In a further aspect of the present disclosure, in some embodiments the example system described above includes a temperature-controlled housing, for housing the plurality of gamma detector assemblies and maintaining the plurality of gamma detector assemblies at a set temperature. The Applicant has discovered that the plurality of gamma detector assemblies, comprising at least the sodium iodine crystals operatively coupled to the PMTs, are susceptible to recording gamma ray spectra comprising peaks that are characteristic of particular elements wherein the peaks are shifted by degrees, and that these observed spectral shifts are due to a change in detector gain with changes in temperature. As each detector assembly is slightly different and requires calibration, it would be difficult to obtain a calibration coefficient to account for these changes in detector gain based on changes in temperature. Thus, the Applicant discovered that housing the detector assembly or plurality of detector assemblies (including the sodium iodine crystal and the PMT of each detector assembly) in a temperature-controlled environment eliminated the observed shifts in the characteristic spectral peaks on the measured gamma spectra. In one example embodiment, the detector assembly temperatures are held stable at a selected temperature within +/−0.25° C. in order to obtain a peak stability of approximately +/−0.5%.

In another aspect of the present disclosure, a hydrogen peak coefficient is obtained for automatically correcting an acquired gamma spectra to account for the moisture content in a soil, which results in the attenuation of the fast neutrons by the Hydrogen atoms in the water molecules that are present in the soil, thereby affecting the peak area of the characteristic gamma peaks used to measure the other elements present in the soil, such as Carbon or Silicon. By applying the hydrogen peak coefficient to correct an acquired gamma spectrum obtained for soil across a geographic area, a more accurate determination of the targeted element in the soil may be obtained, particularly where the content of the targeted element and/or the moisture content of the soil is relatively high. In an illustrative example provided herein, the hydrogen peak coefficients are obtained from simulated soil calibration blocks run through a Monte Carlo modelling simulation, and the hydrogen peak coefficients are then used to derive an equation for using the net peak areas of the characteristic silicon and carbon peaks, from an acquired gamma spectra for an actual soil sample, and in combination with a measurement of the moisture content of that soil sample, determining the carbon content of that soil sample. Although the illustrative example provided herein is applied to the determination of carbon content in the soil, it will be appreciated that the same methods and systems described herein may be applied to obtain more accurate measurements of other elements that may be present in the soil, including but not limited to: Oxygen, Silicon, Iron and Aluminum, and any other elements having characteristic peaks in an inelastic neutron scattering (INS) gamma spectrum.

In yet another aspect of the present disclosure, novel methods and systems are disclosed for obtaining a detailed and accurate map of the moisture content of a soil over a geographical area, as determined from gamma spectra acquired from scanning the soil over that geographical area or a portion thereof. In this aspect, pulsed fast thermal neutron gamma analysis (PFTNA) is utilized to acquire the gamma spectra from the soil under neutron irradiation. Because hydrogen peaks have a clear gamma peak at 2.223 MeV in the thermal neutron capture (TNC) spectrum, due to the thermal neutron reaction, and the area of this peak depends primarily on the amount of water in the soil being scanned, the acquired gamma spectrum is analyzed to determine the water content in the soil.

In another aspect of the present disclosure, the methods and systems described above may be combined in different permutations to obtain a more accurate analysis of the elemental and/or moisture content of the soil. For example, not intended to be limiting, the PFTNA system used to acquire the gamma spectra from scanning the soil over the geographical area of interest (or a portion thereof) may incorporate the temperature-controlled housing, so as to maintain the plurality of gamma detectors at a selected temperature in order to eliminate the observed spectral shifting that may otherwise occur with changes in ambient temperature during soil scanning and gamma spectra acquisition, regardless of whether the methods described herein are employed to determine the soil moisture content and/or the elemental content of a soil.

In one aspect of the present disclosure, methods and systems are provided for measuring the content of an element in a surface layer of a soil of a geographic area. In an embodiment, the system comprises a neutron source for irradiating the surface layer of the soil with neutrons at a location within the geographic area; a detector assembly comprising a plurality of gamma detectors, the detector assembly configured to detect at least an inelastic neutron scattering (INS) gamma spectrum of the surface layer of the soil at the location within the geographic area; an instrument for measuring a moisture content of the surface layer of the soil at the location; and a processor in communication with the detector assembly. The processor may be configured to: associate the detected INS gamma spectrum with the geographic coordinates of the location where the detected INS gamma spectrum was acquired; apply a moisture calibration coefficient to calculate an amount of the element obtained from a net peak area of a characteristic peak of the element in the INS gamma spectrum, the net peak area obtained by subtracting a background peak area of a characteristic peak of the element from a measured peak area of the characteristic peak of the element in the detected INS gamma spectrum, the moisture calibration coefficient calculated to account for the moderation of fast neutrons by a quantity of hydrogen atoms present in the irradiated surface layer of the soil at the location under analysis, the quantity of hydrogen atoms present in the irradiated surface layer of the soil approximated by the moisture content of the surface layer of the soil as measured by the instrument; and generate a concentration of the element in the surface layer of the soil at the location from the amount of the element obtained from the net peak area of the characteristic peak of the element in the INS gamma spectrum.

In some embodiments, the moisture calibration coefficient is calculated from simulated gamma spectra obtained from a simulated soil model, the simulated soil model comprising a plurality of simulated soil samples, each simulated soil sample of the plurality of soil samples containing a composition of elements including at least hydrogen, oxygen, carbon and silicon, the composition of elements in each simulated soil sample varying from the composition of elements in the other simulated soil samples of the plurality of simulated soil samples.

In some embodiments, the moisture calibration coefficient is calculated from a calibration data set, the moisture calibration coefficient normalized by multiplying the moisture calibration coefficient calculated from the calibration data set by a ratio of the value of the moisture calibration coefficient at zero moisture content obtained from the calibration data set divided by the value of the moisture calibration coefficient calculated from the simulated gamma spectra at zero moisture content, the calibration data set comprising a plurality of gamma spectra acquired from a plurality of calibration blocks, each calibration block of the plurality of calibration blocks comprising a known quantity of at least carbon and silicon. In some embodiments, each calibration block of the plurality of calibration blocks contains less than 5% moisture as determined from a moisture measurement of the calibration block.

In some embodiments, the detector assembly is configured to simultaneously detect the INS gamma spectrum and a thermal neutron capture (TNC) gamma spectrum of the surface layer of the soil, and the instrument for measuring the moisture content of the surface layer of the soil comprises the detector assembly. In some embodiments, the element under analysis is selected from a group comprising: carbon, silicon, oxygen, iron, aluminum.

In some embodiments, the system further comprises the neutron source, the detector assembly and the processor mounted to a mobile cart. In such embodiments, the system is configured to measure the content of the element in the surface layer of the soil in a plurality of locations across a geographic area with the processor configured to associate each detected INS and TNC gamma spectra of a plurality of detected INS and TNC gamma spectra with the geographic coordinates of the location where the detected INS and TNC gamma spectra was acquired, and the location is included in a plurality of locations spread across the geographic area. The processor is configured to generate the concentration of the element in the surface layer of the soil for each location of the plurality of locations. In some embodiments, the system further comprises a global positioning system (GPS) and the processor is configured to obtain the geographic coordinates of each location of the plurality of locations from the GPS. The processor may be further configured to generate a map of the geographic area, the map indicating the concentration of the element in the surface layer of the soil for each location of the plurality of locations across the geographic area. The processor may be configured to calculate an average measured peak area of the characteristic peak of the element for a midway point located midway between two adjacent locations of the plurality of locations, the average measured peak area of the characteristic peak of the element calculated from two or more acquired INS and TNC gamma spectra obtained at each location of the two adjacent locations and between the two adjacent locations. In some embodiments, the processor may be configured to generate a map of the geographic area, the map indicating the concentration of the element in the surface layer of the soil across the geographic area, the concentration of the element in the surface layer of the soil obtained from calculating the concentration of the element at each midway point based upon the average measured peak area associated with each midway point between two adjacent locations of the plurality of locations.

In some embodiments, the detector assembly of the system may be enclosed in a temperature-controlled housing, the temperature-controlled housing comprising a temperature sensor for detecting a temperature of the detector assembly, the temperature sensor in communication with a temperature controller, the temperature controller for receiving signals from the temperature sensor and actuating a heating unit to heat an interior of the housing when the temperature sensor detects a temperature of the detector assembly is less than a target temperature.

In another aspect of the present disclosure, a method and system are provided for measuring a content of water in a surface layer of a soil of a geographic area. In some embodiments, the system comprises: a neutron source for irradiating the surface layer of the soil with neutrons at a location of a plurality of locations within the geographic area; a detector assembly comprising a plurality of gamma detectors, the detector assembly configured to detect at least a TNC gamma spectrum of the surface layer of the soil at the location of the plurality of locations within the geographic area; a processor in communication with the detector assembly, the neutron source, the detector assembly and the processor mounted to a mobile cart. The processor may be configured to, for each location of the plurality of locations: associate the detected TNC gamma spectrum with the geographic coordinates of the location where the TNC gamma spectrum was acquired; calculate a net hydrogen peak area of a hydrogen peak of the detected TNC spectrum having a centroid at 2.223 MeV, the net hydrogen peak area calculated by subtracting a background hydrogen peak area from a measured hydrogen peak area obtained from the detected TNC gamma spectrum acquired at the location; and apply a moisture calibration equation to the net hydrogen peak area to generate a soil water content of the surface layer of the soil at the location, the moisture calibration equation providing a quantitative relationship between the calculated net hydrogen peak area and the corresponding soil water content of the surface layer of the soil. In some embodiments, the systems and methods may be configured to calculate a soil gravimetric water content or a soil volumetric water content of the surface layer of the soil at each location of the plurality of locations.

In some embodiments, the moisture calibration equation is obtained from a hydrogen calibration data set, the hydrogen calibration data set comprising a plurality of TNC gamma spectra acquired by the system from a plurality of calibration blocks. Each block of the plurality of calibration blocks contains known quantities of hydrogen, silicon and carbon, wherein the known quantities of hydrogen, silicon and carbon in each calibration block varies from the known quantities of hydrogen, silicon and carbon in each of the other calibration blocks in the plurality of calibration blocks. In some embodiments, the quantities of hydrogen, silicon and carbon in each calibration block of the plurality of hydrogen calibration blocks is provided by a mixture of dry sand and dry polyethylene powder. The mixture of dry sand and dry polyethylene powder of each calibration block may have a moisture content of less than 5% prior to acquiring the TNC gamma spectra of each calibration block of the plurality of calibration blocks.

In some embodiments, the system further comprises a global positioning system (GPS) wherein the processor is configured to obtain the geographic coordinates of each location of the plurality of locations from the GPS. In some embodiments, the processor is further configured to generate a map of the geographic area, the map indicating the soil water content of the surface layer of the soil for each location of the plurality of locations across the geographic area. The processor may be, in some embodiments, configured to calculate an average net hydrogen peak area for a midway point located midway between two adjacent locations of the plurality of locations, the average net hydrogen peak area calculated from two or more acquired TNC gamma spectra obtained at each location of the two adjacent locations and between the two adjacent locations. The processor may be further configured to generate a map of the geographic area, the map indicating the soil water content of the surface layer of the soil across the geographic area, the soil water content of the surface layer of the soil obtained from calculating the soil water content at each midway point based upon the average net hydrogen peak area associated with each midway point between two adjacent locations of the plurality of locations.

In some embodiments, the detector assembly may be enclosed in a temperature-controlled housing, the temperature-controlled housing comprising a temperature sensor for detecting a temperature of the detector assembly, the temperature sensor in communication with a temperature controller, the temperature controller for receiving signals from the temperature sensor and actuating a heating unit to heat an interior of the housing when the temperature sensor detects a temperature of the detector assembly is less than a target temperature.

In another aspect of the present disclosure, a system for measuring a concentration of at least one element in a surface layer of a soil in a geographic area is provided. The system comprises: a detector assembly comprising a plurality of gamma detectors, each gamma detector of the plurality of gamma detectors comprising a sodium iodine crystal coupled to a photomultiplier tube, each gamma detector of the plurality of gamma detectors configured to detect at least one gamma ray spectrum of each location of a plurality of locations across the geographic area. The detector assembly may be enclosed in a temperature-controlled housing, the temperature-controlled housing comprising a temperature sensor for detecting a temperature of the detector assembly, the temperature sensor in communication with a temperature controller, the temperature controller for receiving signals from the temperature sensor and actuating a heating unit, the heating unit actuated to heat an interior of the housing when the temperature sensor detects a temperature of the detector assembly is less than a target temperature. The system may further comprise a processor in communication with the plurality of gamma detectors, the processor configured to: associate a gamma ray spectrum with a geographic coordinates of each location of the plurality of locations where the gamma ray spectrum was acquired; and calculate a concentration of the at least one element at each location of the plurality of locations within the geographic area based on a net peak area of a characteristic peak of the at least one element obtained from the acquired gamma spectrum.

In some embodiments of the system, the temperature controller may be configured to actuate a cooling unit to cool an interior of the housing when the temperature sensor detects a temperature of the detector assembly is greater than a target temperature. In some embodiments, the target temperature is 20° C. In some embodiments, the system is configured to maintain the target temperature within a range of greater than or less than 0.5° C. to maintain a peak stability of greater than or less than 1% of the centroid of the characteristic peak of the at least one element. In some embodiments, the system is configured to maintain the target temperature within a range of greater than or less than 0.25° C. to maintain a peak stability of greater than or less than 0.5% of the centroid of the characteristic peak of the at least one element.

### BRIEF DESCRIPTION OF THE DRAWINGS

The concepts described herein are illustrated by way of example and not intended to be limited to the illustrative examples described below and illustrated in the accompanying figures. For simplicity and clarity of illustration, elements illustrated in the figures are not necessarily drawn to scale. For example, the dimensions of some elements may be exaggerated relative to other elements for clarity. Further, where considered appropriate, reference labels have been repeated among the figures to indicate corresponding or analogous elements. The detailed description particularly refers to the accompanying figures in which:

FIG. 1A is a top plan view of a computer simulation model of the sand-carbon mixtures and the soil neutron stimulated gamma spectra, including the main components of a mobile

PFTNA system for acquiring the gamma spectra, including the wheels of the cart, the shielding around the components of the PFTNA system and the detectors, showing the gamma flux in gamma/cm²/second at an irradiation of 1×10⁷ neutrons/second.

FIG. 1B is a side plan view of the computer simulation model of FIG. 1A.

FIG. 1C is a front plan view of the computer simulation model of FIG. 1A.

FIG. 2A is a computer simulated gamma spectrum of a simulated soil sample.

FIG. 2B is a measured gamma spectrum taken of a neutron-stimulated soil sample.

FIG. 3A is an example of a Gaussian peak area approximation of a silicon peak.

FIG. 3B is an example of a Gaussian peak area approximation of a carbon peak.

FIG. 4A is a computer simulated gamma spectra of a plurality of simulated calibration pits, showing the full gamma ray energy range.

FIG. 4B is a close-up view of the characteristic carbon peaks of the computer simulated gamma spectra of FIG. 4A.

FIG. 4C is a close-up view of the characteristic silicon peaks of the computer simulated gamma spectra of FIG. 4A.

FIG. 5A is a neutron stimulated gamma spectra of a plurality of sand-carbon calibration pits, showing the full gamma ray energy range.

FIG. 5B is a close-up view of the characteristic carbon peaks of the neutron stimulated gamma spectra of FIG. 5A.

FIG. 5C is a close-up view of the characteristic silicon peaks of the neutron stimulated gamma spectra of FIG. 5A.

FIG. 6 is a graph showing the calibration dependencies for different soil moisture contents in a plurality of sand-carbon calibration pits.

FIG. 7 is a graph plotting the calibration coefficients k1 and k2 with their corresponding soil moisture wt % and their corresponding equations.

FIG. 8A is a computer simulated gamma spectra providing the full gamma ray energy range of a plurality of simulated calibration pits, the calibration pits including layers of soil with different moisture contents.

FIG. 8B is a close-up view of the characteristic carbon peaks of the computer simulated gamma spectra of FIG. 8A.

FIG. 8C is a close-up view of the characteristic silicon peaks of the computer simulated gamma spectra of FIG. 8A.

FIG. 9A is a schematic diagram of an embodiment of a mobile PFTNA system.

FIG. 9B is a photograph of an embodiment of a mobile PFTNA system.

FIG. 10 is an example of a gamma spectra of a soil acquired by a mobile PFTNA system, illustrating both the INS and the TNC spectra.

FIG. 11 is a portion of a gamma spectrum, showing the hydrogen peak in a TNC spectrum for PFTNA measurements taken at different soil locations.

FIG. 12 is a pair of graphs, illustrating an example of hydrogen peak fitting by taking the sum of two Gaussian curves to obtain the corrected hydrogen peak area.

FIG. 13 is a graph plotting the relationship between the hydrogen peak area of a soil location as measured by a PFTNA system and the height of the PFTNA system above the ground.

FIG. 14 is a map illustrating the locations of the points of moisture measurement taken with instrumentation and the weighted centers of moisture measurement obtained with a PFTNA system traversing across a field.

FIG. 15 is a graph plotting the relationship between the measured net hydrogen peak area acquired from gamma spectra and the soil gravimetric water content at that same position on a field as obtained from instrumentation measurements for a plurality of fields.

FIG. 16 is a graph plotting the relationship between the soil gravimetric water content as measured by instrumentation and the net hydrogen peak area acquired from gamma spectra and a calibration line obtained from the plot.

FIGS. 17A and 17B are soil moisture distribution maps of the Camp Hill field, with the distribution map of FIG. 17A generated from gamma spectra obtained from a PFTNA system and the distribution map of FIG. 17B generated from moisture measurements obtained by a TDR 350 gauge.

FIGS. 17C and 17D are soil moisture distribution maps of the Field 7 field, with the distribution map of FIG. 17C generated from gamma spectra obtained from a PFTNA system and the distribution map of FIG. 17D generated from moisture measurements obtained by a TDR 350 gauge.

FIG. 18 is a perspective view of an embodiment of a PFTNA system, with the gamma detector assembly of the PFTNA system contained within a temperature-controlled housing.

### DETAILED DESCRIPTION

While the concepts of the present disclosure are susceptible to various modifications and alternative forms, specific embodiments thereof have been shown by way of example in the figures and will be described herein in detail. It should be understood, however, that there is no intent to limit the concepts of the present disclosure to the particular examples or embodiments disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

The disclosed embodiments may be implemented, in some cases, in hardware, firmware, software, or any combination thereof. The disclosed embodiments may also be implemented as instructions carried by or stored on a transitory or non-transitory computer-readable storage medium, which may be read and executed by one or more processors. A computer-readable storage medium may be embodied as any storage device, mechanism, or other physical structure for storing or transmitting information in a form readable by a computing device (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

### Hydrogen Peak Coefficients—Correcting for Soil Moisture Content

Neutron gamma analysis makes it possible to define soil carbon content on the basis of measuring the neutron stimulated gamma spectra of soil. Based on calculations of the main peak areas of interest in the gamma spectra (e.g., silicon and carbon peaks) and using a previously defined calibration dependency (i.e., dependency of the peak areas versus carbon content in reference samples), carbon content in soil can be determined. For example, the determination of carbon content in soil, utilizing a mobile PFTNA apparatus, is described in U.S. Pat. No. 11,397,277, which is incorporated herein by reference.

However, the presence of water in soil can affect peak area values of other elements in soil, such as carbon or silicon, in the inelastic neutron scattering (INS) spectra due to the high moderation property of hydrogen. Some of the irradiating fast neutrons are moderated by hydrogen atoms in soil, thus removing them from the fast neutron energy state and preventing them from activating INS reactions. The number of moderated neutrons depends on the amount of hydrogen atoms in the soil under analysis. Therefore, the effect on the INS reactions caused by the presence of water in the soil (which is the primary source of hydrogen atoms in soil), ought to be defined to account for the moderating of fast neutrons by hydrogen atoms (or water) present in soil, to thereby obtain a more accurate measurement of other elements, such as carbon and silicon content in soil, via neutron gamma analysis.

To measure the effect of water present in soil on gamma spectra obtained from neutron gamma analysis, it is difficult to prepare calibration blocks containing known amounts of hydrogen due to the presence of water, because water may either evaporate into the atmosphere or adsorb onto the surface of the calibration block. This means that the exact amount of water within the block is constantly changing with time. For the same reasons, it is difficult to thoroughly mix the materials within the calibration block to obtain a homogenous sample having a consistent moisture content throughout the sample. Due to these and other difficulties in preparing large soil samples (such as, calibration blocks) with known moisture content, which samples may be several cubic meters weighing several metric tons, the Applicant performed computer simulations of the neutron stimulated gamma spectra of soil using a specialized computer program named the Monte Carlo N-Particle or “MCNP” (Werner, Christopher John, Bull, Jeffrey S., Solomon, C. J., Brown, Forrest B., Mckinney, Gregg Walter, Rising, Michael Evan, Dixon, David A., Martz, Roger Lee, Hughes, Henry G., Cox, Lawrence James, Zukaitis, Anthony J., Armstrong, J. C., Forster, Robert Arthur, and

Casswell, Laura. MCNP Version 6.2 Release Notes. United States: N. p., 2018. Web. doi: 10.2172/1419730).

### Computer Simulations of Gamma Spectra

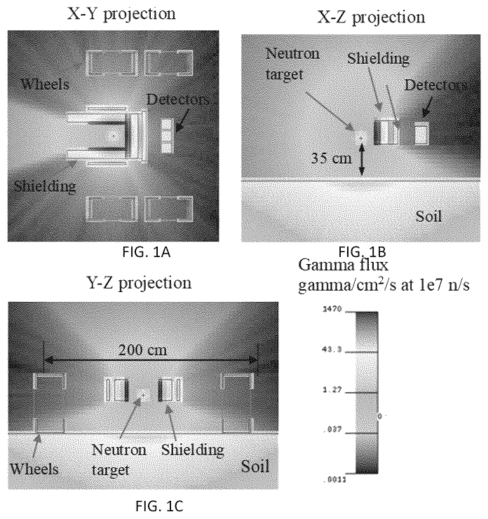

MCNP simulations of neutron stimulated soil gamma spectra were performed to analyze the effect of soil moisture on the determination of soil carbon content using INS gamma spectra analysis. The developed MCNP model used for simulating the neutron irradiated soil gamma spectra is shown in FIG. 1. This model reproduces the main features of the experimental cart used for soil carbon measurements in the field. In particular, this included sizes and disposition of main system components (i.e., neutron source, gamma detectors, shielding and chassis, and sizes of calibrations pits used for actual measurements).

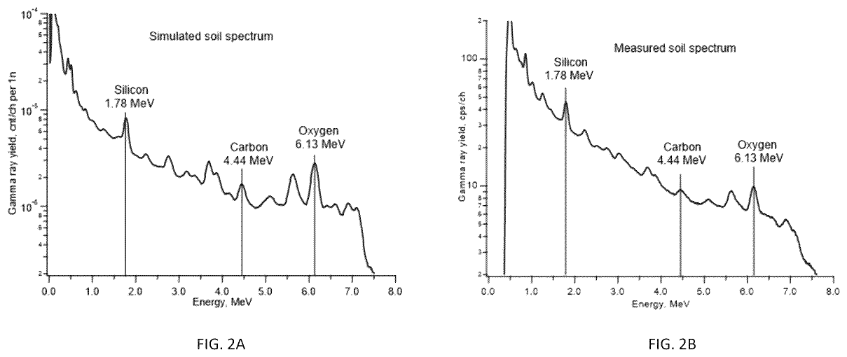

To validate the MCNP model simulations of gamma spectra obtained from a soil sample, a computer simulation and a measured spectra were each obtained from a soil sample containing known amounts of carbon, silicon and oxygen. A comparison of simulated and measured spectra is shown in FIG. 2. As viewed in FIG. 2, the simulated and measured gamma spectra are very similar, with both spectra situated in the same energy range. The characteristic gamma peaks (e.g., silicon with a peak centroid at 1.78 MeV, carbon with a peak centroid at 4.44 MeV, and oxygen with a peak centroid at 6.13 MeV) are present in both spectra and are practically equal in width and relative height. This result demonstrates the feasibility of utilizing the developed MCNP soil model and the simulation method used to create gamma spectra for analyzing the presence of water in soil.

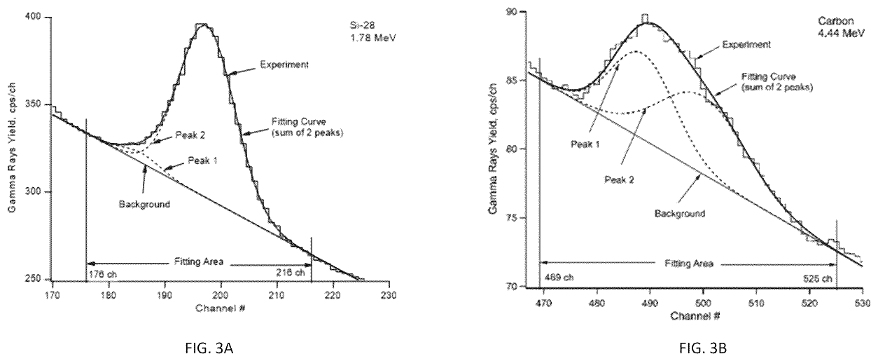

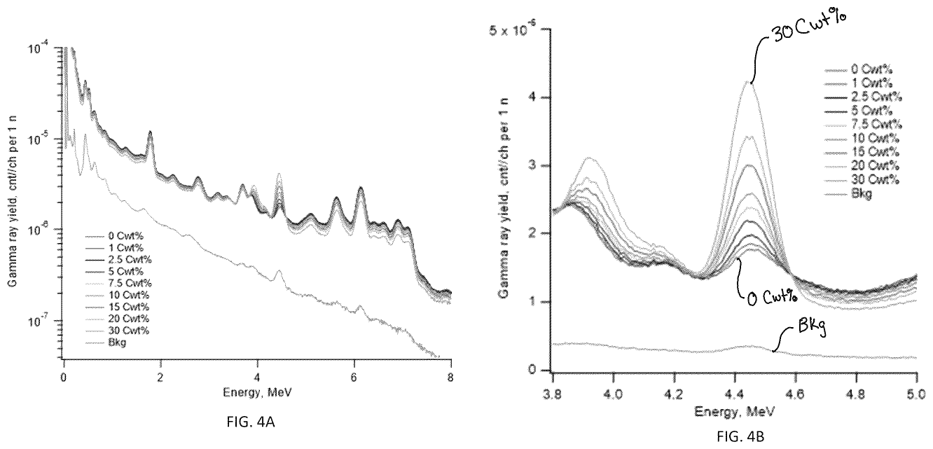

It is assumed that each peak in the neutron stimulated soil gamma spectrum is associated with some element present in both the soil and surrounding objects. The value of a particular peak area reflects the amount of a certain element. In particular, a peak with a centroid at 4.44 MeV is associated with the amount of carbon in soil, as well as carbon present in materials of the measurement system such as components of the PFTNA system mounted to a mobile cart. In addition, the silicon cascade transition peak with a centroid at 4.50 MeV overlaps the carbon peak. The soil carbon content in soil in weight percent (Cwt % soil) may be determined as follows:

$$Cwt {\%}_{s o i l} = \frac{( {C\_pa}_{s o i l} - {C\_pa}_{b k g} ) - k 1 \cdot ( {Si\_pa}_{s o i l} - {Si\_pa}_{b k g} )}{k 2} \qquad ( 1 )$$

where C_paₛₒᵢₗ and C_pa_{bkg} are carbon peak areas (centroid at 4.44 MeV) in the gamma spectra of the soil and in the background, respectively (i.e. gamma spectra measured without samples); and where Si_paₛₒᵢₗ and Si_pa_{bkg} are silicon peak areas (centroid at 1.78 MeV) in the gamma spectra of the soil and in the background, respectively. Whereas, k1 and k2 are calibration coefficients determined for the element under analysis, which in the example of equation (1) above, is carbon. Peak area values may be calculated by approximating with Gaussian(s). Software, such as Igor Pro software published in 2017 by WaveMetrics™, was used for such Gaussian approximations; examples of such Gaussian approximations are illustrated in FIGS. 3A-3B. For example, a Gaussian approximation of the area of a Silicon peak is shown in FIG. 3A and a Gaussian approximation of the area of a Carbon peak is shown in FIG. 3B.

The area between the approximation curve and base line shown in FIGS. 3A and 3B may be calculated by standard equations of Gaussian area. The resulting calculated value may be accepted as the peak area for each curve.

Calibration coefficients k1 and k2 for each element to be analyzed may be obtained from calibration procedures. For the calibration procedure, measurements or simulations may be performed using several reference samples (soil or specially prepared pits) with known amounts of the element to be analyzed, such as the amount of the element carbon content in the soil (Cwt %_{pit,i}). Optionally, rather than utilizing calibration pits, Monte-Carlo simulations may be performed to obtain gamma spectra of samples with known carbon content. Silicon and carbon peak areas may be calculated from these spectra. From peak areas values of several reference samples, which are obtained (for example) from either simulated spectra or spectra measured from a plurality of calibration pits, calibration coefficients may be determined (for example, see Yakubova, G., A. Kavetskiy, S.A. Prior, and H. A. Torbert. 2017. Applying Monte Carlo simulations to optimize an inelastic neutron scattering system for soil carbon analysis. Applied Radiation and Isotopes. 128:237-248. http://dx.doi.org/10.1016/j.apradiso.2017.07.003).

### Model of Carbon Calibration Pits—Dry

Gamma spectra simulations utilizing the design model shown in FIG. 1 were performed using simulated calibration pits sized 400 cm×400 cm×60 cm. Sand-carbon mixtures were used as the sample material. Elemental contents in dry sand-carbon mixtures were defined according to the following calculations.

The elemental content of Si and O in dry sand (SiO₂) was calculated as wt % Si_{sand}=46.7% and wt % O_{sand}=53.3% using the following equations:

$$wt \% {Si}_{s a n d} = \frac{Aw Si}{Mw SiO 2} \qquad ( 2 )$$

$$wt \% {O}_{s a n d} = \frac{Aw O}{Mw SiO 2} \qquad ( 3 )$$

where wt % Si_{sand} and wt % O_{sand} are the weight percent of silicon and oxygen in sand, respectively, AwSi is the atomic weight of silicon (˜28 amu), AwO is the atomic weight of oxygen (˜16 amu), and MwSiO₂ is the molecular weight of sand (˜60 amu).

The elemental content in dry sand-carbon mixtures was calculated as:

$$wt \% Si O {2}_{m i x , d r y} = 100 - wt \% {C}_{m i x , d r y} \qquad ( 4 )$$

$$wt \% {Si}_{m i x , d r y} = wt \% {Si}_{s a n d} \cdot \frac{100 - wt \% {C}_{m i x , d r y}}{100} \qquad ( 5 )$$

$$wt \% {O}_{m i x , d r y} = wt \% {O}_{s a n d} \cdot \frac{100 - wt \% {C}_{m i x , d r y}}{100} \qquad ( 6 )$$

where wt % SiO2_{mix,dry}, Wt % Si_{mix,dry}, Wt % O_{mix,dry}, wt % C_{mix,dry} are the weight percent of sand, silicon, oxygen, and carbon in sand-carbon mixtures, respectively. Simulations were then performed to obtain gamma spectra for wt % C_{mix,dry} were 0, 1, 2.5, 5, 7.5, 10, 15, 20, and 30 wt % C, using the MCNP software.

Densities of the above-noted dry sand mixtures were calculated as:

$$( 7 )$$

$${d}_{m i x , d r y} ( wt \% {C}_{m i x , d r y} ) = \frac{100 \cdot 1.7 \cdot 0.53}{1.7 \cdot wt \% {C}_{m i x , d r y} + 0.53 \cdot wt \% SiO {2}_{m i x , d r y}} , g / {cm}^{3}$$

where 1.7 g/cm3 is the sand density, (with the sand comprising the elements silicon and oxygen in the calibration pit), and 0.53 g/cm3 is density of the carbon material (in this example, without intending to be limiting, coconut shell was used in the calibration mixtures to add the known carbon content of the mixture). The elemental content and densities of dry mixtures used in simulations are presented in Table 1.

TABLE 1
Elemental content and densities of dry sand-carbon mixtures.

| wt % C_{mix, dry} | 0 | 1 | 2.5 | 5 | 7.5 | 10 | 15 | 20 | 30 |
|---|---|---|---|---|---|---|---|---|---|
| wt % SiO2_{mix, dry} | 100 | 99 | 97.5 | 95 | 92.5 | 90 | 85 | 80 | 70 |
| Mixture density, g/cm³ | 1.70 | 1.66 | 1.61 | 1.53 | 1.46 | 1.39 | 1.28 | 1.18 | 1.02 |
| wt % Si_{mix, dry} | 46.7 | 46.3 | 45.6 | 44.4 | 43.2 | 42.1 | 39.7 | 37.4 | 32.7 |
| wt % O_{mix, dry} | 53.3 | 52.7 | 51.9 | 50.6 | 49.3 | 47.9 | 45.3 | 42.6 | 37.3 |

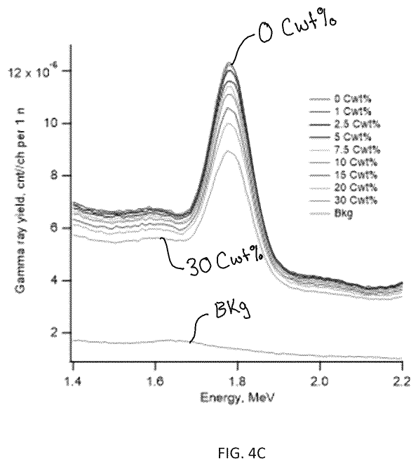

FIGS. 4A to 4C provide gamma spectra of dry sand-carbon calibration pits based on the computer model simulations, including system background spectra. FIG. 4A is a gamma spectra of the full energy range, with the simulated dry sand-carbon calibration pits ranging from containing 0% Carbon by weight to 1%, 2.5%, 5%, 7.5%, 10%, 15%, 20% and 30% Carbon by weight, respectively, and the gamma spectrum for the background is also shown. FIG. 4B is a close-up view of the gamma spectra of FIG. 4A, showing the peaks characteristic of Carbon, and FIG. 4C is a close-up view of the gamma spectra of FIG. 4A, showing the peak characteristic of Silicon. As may be appreciated from FIGS. 4A to 4C, the peak area of the characteristic Carbon peaks (i.e. the peak with a centroid at 4.44 MeV) increased as carbon content increased, while the peak area of the characteristic Silicon peaks (i.e. the peak with a centroid at 1.78 MeV) decreased as carbon content increased. The simulated gamma spectra of the dry sand-carbon calibration pits therefore correspond to what would be expected of measured gamma spectra obtained from the calibration pits, the contents of which are described in Table 1 above.

Spectra peak areas of the gamma spectra provided in FIGS. 4A to 4C were determined by Gaussian approximation. Net peak areas of carbon and silicon, shown in Table 2 below, were calculated using the following equations:

$$netCpa = {Cpa}_{s o i l} - {Cpa}_{b k g} \qquad ( 8 )$$

$$netSipa = {Sipa}_{s o i l} - {Sipa}_{b k g} \qquad ( 9 )$$

where the net peak area of the carbon peak, netCpa, is calculated by subtracting the background total peak area of the characteristic Carbon peak from the total area of the characteristic Carbon peak. Likewise, the net peak area of the silicon peak, netSipa, is calculated by subtracting the background total peak area of the characteristic Silicon peak from the total area of the characteristic Silicon peak.

TABLE 2
Net peak areas (“pa”) of silicon
(error ± 0.5 cnt/1n) and carbon
(error ± 0.2 cnt/1n) peaks from simulated gamma
spectra of dry sand-carbon calibration pits.

| wt % C_{mix, dry} | 0 | 1 | 2.5 | 5 | 7.5 | 10 | 15 | 20 | 30 |
|---|---|---|---|---|---|---|---|---|---|
| netSipa, cnt/1n, ×10⁻⁶ | 90.5 | 88.7 | 87.6 | 84.6 | 82.8 | 79.6 | 73.9 | 68.4 | 58.5 |
| netCpa, cnt/1n, ×10⁻⁶ | 9.6 | 11.5 | 13.4 | 17.1 | 21.2 | 25.0 | 32.5 | 39.6 | 54.9 |

### Calibration Dependency

The calibration dependency used for determining the weight percentage of carbon in soil, wt % Cₛₒᵢₗ, is defined as the dependency of the carbon peak area associated with carbon only (netC_pa_{pit,i}-k1. netSi_pa_{pit,i}) as compared to the portion of the peak area that is contributed to by the presence of silicon, due to the silicon cascade transition peak with a centroid at 4.50 MeV which overlaps the carbon peak with a centroid at 4.44 MeV. In particular, calibration coefficients k1 and k2 in Equation 1 may be determined, based on the respective net peak areas of Carbon and Silicon from the simulated gamma spectra of the dry-sand calibration pits provided in FIGS. 4A to 4C. In performing these calculations, the Applicant assumes that the same carbon content (in weight percent) in both the dry sand-carbon mixture and in a dry soil sample to be measured, would produce the same carbon peak value, and that this value is directly proportional to the carbon content in a soil pit (˜ k2. Cwt %_{pit,i}). Based on these assumptions, the calibration dependency may be determined by using the net peak area data for Si and C from Table 2. In performing these calculations, the least square method may be utilized for determining calibration dependency coefficients. According to this method, the sum of square differences should be minimalized:

$${\sum }_{i} {( {netCpa}_{p i t , i} - k 1 \cdot {netSipa}_{p i t , i} - k 2 \cdot C wt {\%}_{p i t , i} )}^{2} \to min \qquad ( 10 )$$

Derivations of k1 and k2 are found and made equal to zero. Solving this system of two equations with two unknown variables (k1 and k2) then yields the values of k1 and k2. Using data from Table 2 returns values of (8.6±0.2)·10⁻² and (1.60±0.03)·10⁻⁶ for k1 and k2, respectively.

### Validation of Calibration Coefficients

The above-described method for determining calibration coefficients was validated by simulating dry soil spectra, determining the carbon content, and comparing the received value to the carbon content used in the simulation model. This validation testing used modeled soil volumes having homogeneous elemental content and with heterogeneous soil (i.e. where the elemental content of the soil changes with soil depth).

### Homogeneous Soil Volume Validation Testing

In the first of two validation tests of the above-described method for determining calibration coefficients, the contents of the dry homogeneous soil volume (400 cm×400 cm×60 cm) are presented in Table 3. The soil elemental content (other than carbon), with carbon set at wt %=0, was taken as the content for a Mollisols soil, which is a type of soil present in the United States and having available data about this soil type's content in literature (Handbook of Soil Science. 2000. Ed.-in-chief: M. E. Sumner. CRC Press LLC, Boca Raton, London, New York, Washington, D.C. ISBN 0-8493-3136-6). Thus, the soil content for each element of a Mollisols soil, except with carbon set at 0% weight, is obtained from the literature references and is set out in the table below. Then, for each percentage of carbon content in the soil (ie: for the weight of carbon content ranging from 1% to 30%), the elemental content (El) for each non-carbon element, in soils with carbon presence, was calculated as:

$$wt \% El ( wt \% C ) = wt \% El ( 0 ) \cdot ( 1 - \frac{wt \% C}{100} ) \qquad ( 11 )$$

TABLE 3
Soil volume with homogeneous elemental content. Soil density is 1.25 g/cm³.

| Element symbol | Elemental content in wt % | | | | | | | | |
|---|---|---|---|---|---|---|---|---|---|
| C | 0 | 1 | 2.5 | 5 | 7.5 | 10 | 15 | 20 | 30 |
| Si | 36.0 | 35.6 | 35.1 | 34.2 | 33.3 | 32.4 | 30.6 | 28.8 | 25.2 |
| Na | 1.0 | 0.9 | 0.9 | 0.9 | 0.9 | 0.9 | 0.8 | 0.8 | 0.7 |
| Al | 6.4 | 6.4 | 6.3 | 6.1 | 5.9 | 5.8 | 5.5 | 5.1 | 4.5 |
| K | 1.7 | 1.6 | 1.6 | 1.6 | 1.5 | 1.5 | 1.4 | 1.3 | 1.2 |
| Ca | 1.2 | 1.2 | 1.1 | 1.1 | 1.1 | 1.1 | 1.0 | 0.9 | 0.8 |
| Fe | 2.7 | 2.7 | 2.7 | 2.6 | 2.5 | 2.5 | 2.3 | 2.2 | 1.9 |
| Mg | 0.3 | 0.3 | 0.3 | 0.3 | 0.3 | 0.3 | 0.3 | 0.3 | 0.2 |
| O | 50.7 | 50.2 | 49.5 | 48.2 | 46.9 | 45.7 | 43.1 | 40.6 | 35.5 |

The simulated soil spectra obtained for the simulated soil pits, above, appeared similar to the spectra shown in FIGS. 4A to 4C. The defined peak areas with centroids at 1.78 and 4.44 MeV and the soil carbon content defined by equation (1) with the above-mentioned coefficient values (k1=(8.6±0.2)·10⁻² and k2=(1.60±0.03)·10⁻⁶ are shown in Table 4. As may be appreciated from the data presented in Table 4, defined values of carbon content (shown in the “C wt % in reference mixture” row of Table 4) practically coincides with the model data (shown in the “Defined C wt %+Error” row of Table 4).

TABLE 4
Comparison of carbon content calculated using calibration coefficients as compared
to reference carbon values (error: netSipa ± 0.5 cnt/1n, netCpa ± 0.2 cnt/1n).

| | C wt % in reference mixture | | | | | | | | |
|---|---|---|---|---|---|---|---|---|---|
| | 0 | 1 | 2.5 | 5 | 7.5 | 10 | 15 | 20 | 30 |
| netSipa, cnt/1n, ×10⁻⁶ | 69.6 | 69.3 | 68.6 | 66.8 | 64.8 | 62.9 | 58.7 | 55.9 | 49.4 |
| netCpa, cnt/1n, ×10⁻⁶ | 6.4 | 7.8 | 10.2 | 13.8 | 17.7 | 21.9 | 29.7 | 37.8 | 54.9 |
| Defined C | 0.3 ± | 1.2 ± | 2.7 ± | 5.0 ± | 7.6 ± | 10.3 ± | 15.4 ± | 20.6 ± | 31.6 ± |
| wt % ± Error | 0.2 | 0.2 | 0.2 | 0.2 | 0.2 | 0.2 | 0.3 | 0.4 | 0.6 |

### Heterogenous Soil Volume Validation Testing

In the second of two validation tests of the above-described method for determining calibration coefficients, a dry soil model is provided where the elemental content of the soil changes with depth, similar to real heterogenous soils. The elemental content of this dry soil model is shown in Table 5. The depth dependence of the soil's constituent elements corresponds to the carbon content depth dependencies for Mollisols provided in literature (Handbook of Soil Science. 2000. Ed.-in-chief: M. E. Sumner. CRC Press LLC, Boca Raton, London, New York, Washington, D.C. ISBN 0-8493-3136-6.)

TABLE 5
Elemental content (wt %) of dry heterogenous soil by depth.

| Element | Soil depth (cm) | | | | | | | | | | | |
|---|---|---|---|---|---|---|---|---|---|---|---|---|
| symbol | 0-2 | 2-4 | 4-6 | 6-8 | 8-10 | 10-12 | 12-15 | 15-20 | 20-30 | 30-40 | 40-50 | 50-60 |
| C | 6.0 | 5.4 | 4.9 | 4.5 | 4.2 | 3.9 | 3.7 | 3.4 | 3.0 | 2.9 | 2.8 | 2.8 |
| Si | 34.2 | 33.9 | 33.7 | 33.4 | 33.1 | 32.8 | 32.4 | 31.8 | 30.8 | 29.7 | 28.8 | 28.0 |
| Na | 0.9 | 0.9 | 0.9 | 0.9 | 0.9 | 0.9 | 0.8 | 0.8 | 0.8 | 0.7 | 0.7 | 0.7 |
| Al | 6.1 | 6.1 | 6.0 | 6.0 | 6.0 | 5.9 | 5.9 | 5.9 | 5.8 | 5.7 | 5.6 | 5.5 |
| K | 1.6 | 1.6 | 1.5 | 1.5 | 1.5 | 1.5 | 1.5 | 1.4 | 1.4 | 1.3 | 1.3 | 1.3 |
| Ca | 1.1 | 1.1 | 1.1 | 1.1 | 1.1 | 1.1 | 1.1 | 1.1 | 1.1 | 1.1 | 9.4 | 9.4 |
| Fe | 2.6 | 2.6 | 2.6 | 2.6 | 2.6 | 2.6 | 2.6 | 2.6 | 2.6 | 2.7 | 2.7 | 2.7 |
| Mg | 0.3 | 0.3 | 0.3 | 0.4 | 0.4 | 0.4 | 0.5 | 0.5 | 0.6 | 0.8 | 0.9 | 1.0 |
| O | 47.2 | 48.1 | 48.9 | 49.6 | 50.3 | 50.9 | 51.5 | 52.4 | 53.8 | 55.2 | 47.9 | 48.6 |

The gamma spectrum for the heterogeneous soil set out in Table 5 was simulated. Received net peak area values were netSipa=(6.65±0.5)·10⁻⁵ cnt/1n and netCpa=(1.35±0.2)·10⁻⁵ cnt/1n. Soil carbon content calculated by equation (1) may be attributed to the value of the average carbon weight percent in the upper 10 cm soil layer. This value was equal to 4.9±0.2 wt %.

For comparison, the average carbon content in the upper 10 cm layer of the soil model described in Table 5 was calculated as:

$$\overline{wt \% C} = \frac{{\sum }_{k = 1}^{k = 5} wt \% {C}_{k} \cdot 2}{10} \qquad ( 12 )$$

where wt % Ck are the carbon contents in the first 5 layers with a thickness of 2 cm each (10 cm total thickness), as set out in Table 5 above. The value obtained from equation (12) was equal to 5.0 wt %. Both values (the average carbon weight percent in the upper 10 cm soil layer received from the simulated gamma spectra and from the soil model described in Table 5) coincided with each other. Thus, the described procedure for determining soil carbon content based on previously defined calibration coefficients may be used to obtain accurate determinations of carbon content in dry soil.

### Defining Carbon Content in Wet Soil

To account for the presence of soil moisture in a soil to be analyzed using gamma spectra to determine the elemental content of the soil, the following steps may be performed. Firstly, the calibration dependencies for soil models having a known carbon content and a known moisture content were defined. Then, simulated gamma spectra were obtained for both homogenous and heterogenous soil models with different moisture levels. Finally, the determination of carbon content using “dry” and “wet with different moistures” calibration dependencies was validated by comparison with values obtained from soil models.

MCNP simulations of gamma spectra of sand-carbon mixtures with different moistures, for creating the calibration dependencies, were performed. The elemental content in wet sand-carbon mixtures were calculated by the following equations:

$$wt \% {Si}_{m i x , w e t} = \frac{wt \% {Si}_{m i x d r y}}{1 + mo / 100} \qquad ( 13 )$$

$$wt \% {C}_{m i x , w e t} = \frac{wt \% {C}_{m i x d r y}}{1 + m o / 100} \qquad ( 14 )$$

$$wt \% {H}_{m i x , w e t} = \frac{m o}{1 + m o / 100} \cdot \frac{2 \cdot Aw H}{Mw H 2 O} \qquad ( 15 )$$

$$wt \% {O}_{m i x , w e t} = \frac{wt \% {O}_{mix , dry}}{1 + m o / 100} + \frac{m o}{1 + m o / 100} \cdot \frac{Aw O}{Mw H 2 O} \qquad ( 16 )$$

where wt % Si_{mix,dry}, Wt % C_{mix,dry}, wt % O_{mix,dry} are data obtained from Table 1; mo is the known moisture % of the soil mixture; AwH=1 and AwO=16 are respective atomic weights of hydrogen and oxygen; and Mw H₂O=18 is the molecular weight of water. The calculated sand-carbon mixture contents with different moistures are presented in Table 6.

TABLE 6
Sand-carbon mixtures with different moisture contents.

| | | | | | | | | | |
|---|---|---|---|---|---|---|---|---|---|
| C wt % in dry mix | 0 | 1 | 2.5 | 5 | 7.5 | 10 | 15 | 20 | 30 |

5% Moisture

| | | | | | | | | | |
|---|---|---|---|---|---|---|---|---|---|
| C wt % in wet mix | 0.0 | 1.0 | 2.4 | 4.8 | 7.1 | 9.5 | 14.3 | 19.0 | 28.6 |
| Si wt % in wet mix | 44.5 | 44.1 | 43.4 | 42.3 | 41.2 | 40.1 | 37.8 | 35.6 | 31.2 |
| H wt % in wet mix | 0.5 | 0.5 | 0.5 | 0.5 | 0.5 | 0.5 | 0.5 | 0.5 | 0.5 |
| O wt % in wet mix | 55.0 | 54.4 | 53.7 | 52.4 | 51.1 | 49.9 | 47.3 | 44.8 | 39.7 |

10% Moisture

| | | | | | | | | | |
|---|---|---|---|---|---|---|---|---|---|
| C wt % in wet mix | 0.0 | 0.9 | 2.3 | 4.5 | 6.8 | 9.1 | 13.6 | 18.2 | 27.3 |
| Si wt % in wet mix | 42.5 | 42.1 | 41.4 | 40.4 | 39.3 | 38.2 | 36.1 | 34.0 | 29.7 |
| H wt % in wet mix | 1.0 | 1.0 | 1.0 | 1.0 | 1.0 | 1.0 | 1.0 | 1.0 | 1.0 |
| O wt % in wet mix | 56.5 | 56.0 | 55.3 | 54.1 | 52.9 | 51.7 | 49.2 | 46.8 | 42.0 |

15% Moisture

| | | | | | | | | | |
|---|---|---|---|---|---|---|---|---|---|
| C wt % in wet mix | 0.0 | 0.9 | 2.2 | 4.3 | 6.5 | 8.7 | 13.0 | 17.4 | 26.1 |
| Si wt % in wet mix | 40.6 | 40.2 | 39.6 | 38.6 | 37.6 | 36.6 | 34.5 | 32.5 | 28.5 |
| H wt % in wet mix | 1.4 | 1.4 | 1.4 | 1.4 | 1.4 | 1.4 | 1.4 | 1.4 | 1.4 |
| O wt % in wet mix | 57.9 | 57.4 | 56.7 | 55.6 | 54.4 | 53.3 | 51.0 | 48.6 | 44.0 |

30% Moisture

| | | | | | | | | | |
|---|---|---|---|---|---|---|---|---|---|
| C wt % in wet mix | 0.0 | 0.8 | 1.9 | 3.8 | 5.8 | 7.7 | 11.5 | 15.4 | 23.1 |
| Si wt % in wet mix | 36.0 | 35.6 | 35.1 | 34.2 | 33.3 | 32.4 | 30.6 | 28.8 | 25.2 |
| H wt % in wet mix | 2.6 | 2.6 | 2.6 | 2.6 | 2.6 | 2.6 | 2.6 | 2.6 | 2.6 |
| O wt % in wet mix | 61.5 | 61.1 | 60.5 | 59.4 | 58.4 | 57.4 | 55.3 | 53.3 | 49.2 |

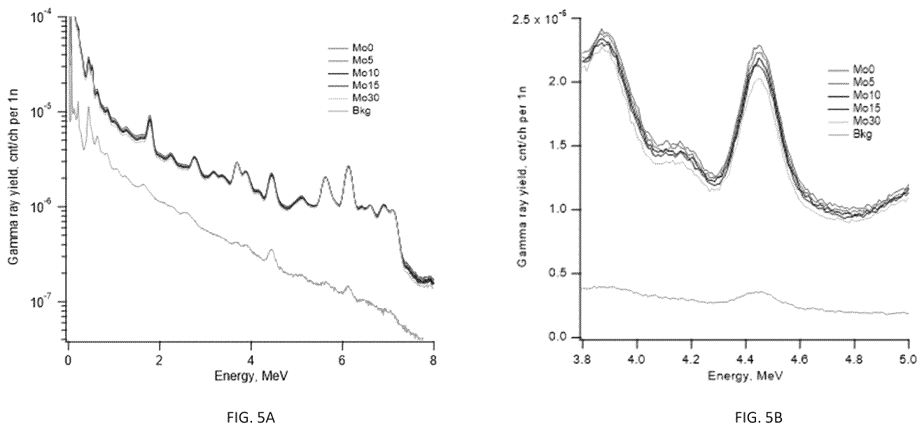

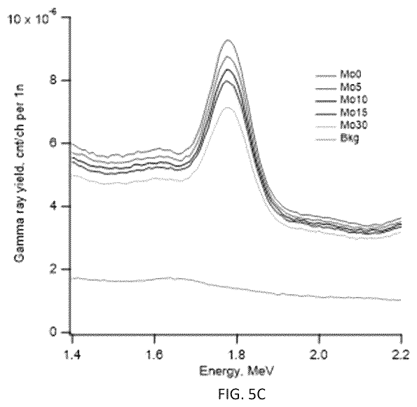

Spectral simulations of wet sand-carbon mixtures were conducted, based on the simulated wet sand-carbon mixtures provided in Table 6. The simulated spectra for 10% carbon content 5 (in dry mix), with different moisture levels, are shown as an example in FIGS. 5A to 5C. FIG. 5A is a gamma spectra of the full energy range, with the simulated wet sand-carbon calibration pits (with 10% carbon by weight at 0% moisture) for different moisture levels, ranging from 0% moisture by weight to 30% moisture by weight. FIG. 5B is a close-up view of the gamma spectra of FIG. 5A, showing the peak characteristic of Carbon, and FIG. 5C is a close-up view of the gamma spectra of FIG. 5A, showing the peak characteristic of Silicon. As may be appreciated from FIGS. 5A to 5C, the peak area of the characteristic Carbon peaks (i.e. the peak with a centroid at 4.44 MeV) slightly decreased as moisture content increased, while the peak area of the characteristic Silicon peaks (i.e. the peak with a centroid at 1.78 MeV) also decreased as the moisture content increased. As the relative amounts of both carbon and silicon decrease with increased moisture levels, as reflected in Table 6, the simulated gamma spectra approximately correspond with the values shown in Table 6.

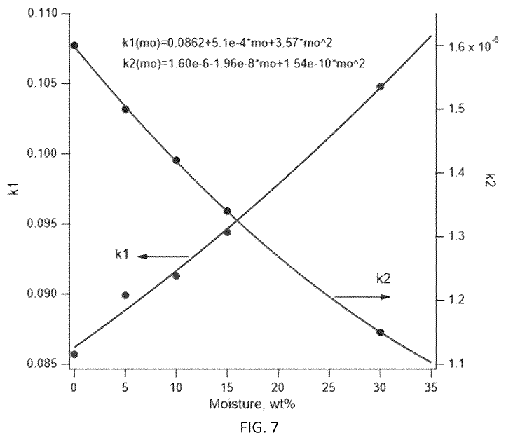

The calculated netSipa and netCpa for the simulated spectra (FIGS. 5A to 5C) of the wet sand-carbon mixtures with different moisture levels, are presented in Table 7. Additionally, the calibration coefficients k1, k2 for carbon are presented in Table 7 for each moisture level. Calibration dependencies with these calibration coefficients for varying moisture levels (ranging from 0-30% moisture) are plotted in FIG. 7. As may be seen, the calibration dependency slope decreases with increasing moisture.

TABLE 7
Net peak areas in spectra of sand-carbon mixtures with different moistures and
calibration coefficients (error: netSipa ± 0.5 cnt/1n, netCpa ± 0.2 cnt/1n).

| | | | | | | | | | |
|---|---|---|---|---|---|---|---|---|---|
| C wt % in dry mix | 0 | 1 | 2.5 | 5 | 7.5 | 10 | 15 | 20 | 30 |

5% Moisture

| | | | | | | | | | |
|---|---|---|---|---|---|---|---|---|---|
| netSipa, cnt/1n, ×10⁻⁶ | 81.6 | 81.1 | 79.6 | 77.2 | 75.1 | 72.0 | 67.2 | 62.6 | 53.5 |
| netCpa, cnt/1n, ×10⁻⁶ | 7.5 | 8.9 | 10.9 | 14.4 | 17.8 | 21.3 | 28.7 | 35.6 | 50.0 |

| | |
|---|---|
| k1 | 0.0899 ± 0.0027 |
| k2 | (1.50 ± 0.05) · 10⁶ |

10% Moisture

| | | | | | | | | | |
|---|---|---|---|---|---|---|---|---|---|
| netSipa, cnt/1n, ×10⁻⁶ | 76.1 | 75.1 | 73.8 | 71.3 | 69.7 | 67.3 | 62.5 | 58.3 | 50.3 |
| netCpa, cnt/1n, ×10⁻⁶ | 7.0 | 8.4 | 10.5 | 13.6 | 16.6 | 20.2 | 27.2 | 33.9 | 47.2 |

| | |
|---|---|
| k1 | 0.0913 ± 0.0027 |
| k2 | (1.42 ± 0.04) · 10⁻⁶ |

15% Moisture

| | | | | | | | | | |
|---|---|---|---|---|---|---|---|---|---|
| netSipa, cnt/1n, ×10⁻⁶ | 71.2 | 70.1 | 68.7 | 67.0 | 64.9 | 63.2 | 59.0 | 54.9 | 47.5 |
| netCpa, cnt/1n, ×10⁻⁶ | 6.7 | 8.0 | 10.0 | 12.9 | 16.1 | 19.3 | 25.6 | 32.2 | 44.5 |

| | |
|---|---|
| k1 | 0.0944 ± 0.0028 |
| k2 | (1.34 ± 0.04) · 10⁻⁶ |

30% Moisture

| | | | | | | | | | |
|---|---|---|---|---|---|---|---|---|---|
| netSipa, cnt/1n, ×10⁻⁶ | 59.9 | 59.3 | 58.4 | 56.9 | 55.5 | 53.7 | 50.0 | 47.2 | 40.5 |
| netCpa, cnt/1n, ×10⁻⁶ | 6.3 | 7.3 | 8.9 | 11.7 | 14.3 | 17.2 | 22.6 | 27.9 | 38.5 |

| | |
|---|---|
| k1 | 0.1048 ± 0.0031 |
| k2 | (1.15 ± 0.03) · 10⁻⁶ |

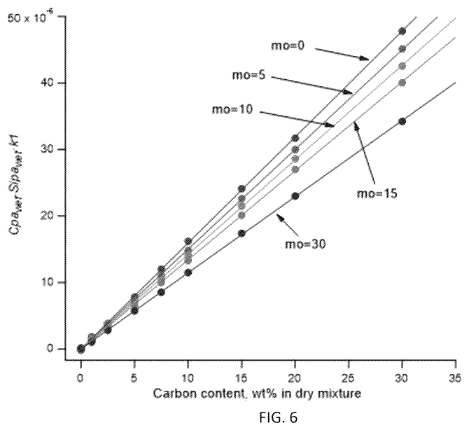

The relationship between calibration coefficients for carbon and moisture are represented by the plot shown in FIG. 6. These dependencies can be approximated by second order polynomials with good agreement. The approximation curves are shown as lines in FIG. 7. Accordingly, the dependencies of coefficients k1 and k2 with moisture mo, %, are:

$$k 1 ( m o ) = 0.0862 + 0.0005103 \cdot + mo + 3.568 \cdot {10}^{- 6} \cdot {mo}^{2} \qquad ( 17 )$$

$$k 2 ( m o ) = 1.5 \; 981 \cdot {10}^{- 6} - 1.9 \; 577 \cdot {10}^{- 8} \cdot mo + 1.5 \; 426 \cdot {10}^{- 10} \cdot {mo}^{2} \qquad ( 18 )$$

Using these polynomial approximations in Equation (1), an equation for calculating soil carbon content (wt % in dry soil) on the basis of silicon and carbon peak area data in the gamma spectra of wet soil (with known moisture, mo %) may be represented as:

$$C wt {\%}_{d r y} = \frac{\begin{matrix}{netCpa}_{w e t} - {netSipa}_{w e t} \cdot \\ ( 0.08 62 + 0.0005 \; 103 \cdot mo + 3.5 \; 68 \cdot {10}^{- 6} \cdot {mo}^{2} )\end{matrix}}{1.5 \; 981 \cdot {10}^{- 6} - 1.9 \; 577 \cdot {10}^{- 8} \cdot mo + 1.5 \; 426 \cdot {10}^{- 10} \cdot {mo}^{2}} \qquad ( 19 )$$

Thus, equation (19) may be used for determination of carbon content in wet soil based on the peak area determination in neutron stimulated gamma spectra. Received values return the carbon weight percent in dry soil.

Several examples were considered for testing the developed equations for determining carbon content in wet soil using neutron induced gamma spectra. In the first example, gamma spectra simulations of a Mollisols with a homogenous sample volume content at different moistures were performed. Elemental content was calculated similar to elemental content calculations of wet sand-carbon mixtures based on data for dry soil (Table 3). The resulting elemental content of wet homogenous soil, at different moisture levels, is presented in Table

TABLE 8
Wet homogenous soil content.

| | C wt % in dry mix | | | | | | | | |
|---|---|---|---|---|---|---|---|---|---|
| | 0 | 1 | 2.5 | 5 | 7.5 | 10 | 15 | 20 | 30 |
| Element symbol | Elemental content, wt %, in wet soil at 5% moisture | | | | | | | | |
| C | 0.0 | 1.0 | 2.4 | 4.8 | 7.1 | 9.5 | 14.3 | 19.0 | 28.6 |
| Si | 34.3 | 33.9 | 33.4 | 32.6 | 31.7 | 30.9 | 29.1 | 27.4 | 24.0 |
| Na | 0.9 | 0.9 | 0.9 | 0.9 | 0.8 | 0.8 | 0.8 | 0.7 | 0.6 |
| Al | 6.1 | 6.1 | 6.0 | 5.8 | 5.7 | 5.5 | 5.2 | 4.9 | 4.3 |
| K | 1.6 | 1.6 | 1.5 | 1.5 | 1.5 | 1.4 | 1.3 | 1.3 | 1.1 |
| Ca | 1.1 | 1.1 | 1.1 | 1.1 | 1.0 | 1.0 | 1.0 | 0.9 | 0.8 |
| Fe | 2.6 | 2.6 | 2.6 | 2.5 | 2.4 | 2.4 | 2.2 | 2.1 | 1.8 |
| Mg | 0.3 | 0.3 | 0.3 | 0.3 | 0.3 | 0.3 | 0.3 | 0.2 | 0.2 |
| H | 0.5 | 0.5 | 0.5 | 0.5 | 0.5 | 0.5 | 0.5 | 0.5 | 0.5 |
| O | 52.5 | 52.1 | 51.3 | 50.1 | 48.9 | 47.7 | 45.3 | 42.9 | 38.1 |
| Element symbol | Elemental content, wt %, in wet soil at 10% moisture | | | | | | | | |
| C | 0.0 | 0.9 | 2.3 | 4.5 | 6.8 | 9.1 | 13.6 | 18.2 | 27.3 |
| Si | 32.7 | 32.4 | 31.9 | 31.1 | 30.3 | 29.5 | 27.8 | 26.2 | 22.9 |
| Na | 0.9 | 0.9 | 0.8 | 0.8 | 0.8 | 0.8 | 0.7 | 0.7 | 0.6 |
| Al | 5.8 | 5.8 | 5.7 | 5.5 | 5.4 | 5.3 | 5.0 | 4.7 | 4.1 |
| K | 1.5 | 1.5 | 1.5 | 1.4 | 1.4 | 1.4 | 1.3 | 1.2 | 1.1 |
| Ca | 1.1 | 1.1 | 1.0 | 1.0 | 1.0 | 1.0 | 0.9 | 0.9 | 0.7 |
| Fe | 2.5 | 2.5 | 2.4 | 2.4 | 2.3 | 2.2 | 2.1 | 2.0 | 1.7 |
| Mg | 0.3 | 0.3 | 0.3 | 0.3 | 0.3 | 0.3 | 0.2 | 0.2 | 0.2 |
| H | 1.0 | 1.0 | 1.0 | 1.0 | 1.0 | 1.0 | 1.0 | 1.0 | 1.0 |
| O | 54.2 | 53.7 | 53.0 | 51.9 | 50.7 | 49.6 | 47.3 | 45.0 | 40.4 |
| Element symbol | Elemental content, wt %, in wet soil at 15% moisture | | | | | | | | |
| C | 0.0 | 0.9 | 2.2 | 4.3 | 6.5 | 8.7 | 13.0 | 17.4 | 26.1 |
| Si | 31.3 | 31.0 | 30.5 | 29.7 | 29.0 | 28.2 | 26.6 | 25.0 | 21.9 |
| Na | 0.8 | 0.8 | 0.8 | 0.8 | 0.8 | 0.7 | 0.7 | 0.7 | 0.6 |
| Al | 5.6 | 5.5 | 5.4 | 5.3 | 5.2 | 5.0 | 4.7 | 4.5 | 3.9 |
| K | 1.4 | 1.4 | 1.4 | 1.4 | 1.3 | 1.3 | 1.2 | 1.2 | 1.0 |
| Ca | 1.0 | 1.0 | 1.0 | 1.0 | 0.9 | 0.9 | 0.9 | 0.8 | 0.7 |
| Fe | 2.4 | 2.4 | 2.3 | 2.3 | 2.2 | 2.2 | 2.0 | 1.9 | 1.7 |
| Mg | 0.3 | 0.3 | 0.3 | 0.3 | 0.3 | 0.2 | 0.2 | 0.2 | 0.2 |
| H | 1.4 | 1.4 | 1.4 | 1.4 | 1.4 | 1.4 | 1.4 | 1.4 | 1.4 |
| O | 55.7 | 55.3 | 54.6 | 53.5 | 52.4 | 51.3 | 49.1 | 46.9 | 42.5 |
| Element symbol | Elemental content, wt %, in wet soil at 30% moisture | | | | | | | | |
| C | 0.0 | 0.8 | 1.9 | 3.8 | 5.8 | 7.7 | 11.5 | 15.4 | 23.1 |
| Si | 27.7 | 27.4 | 27.0 | 26.3 | 25.6 | 24.9 | 23.5 | 22.2 | 19.4 |
| Na | 0.7 | 0.7 | 0.7 | 0.7 | 0.7 | 0.7 | 0.6 | 0.6 | 0.5 |
| Al | 4.9 | 4.9 | 4.8 | 4.7 | 4.6 | 4.4 | 4.2 | 4.0 | 3.5 |
| K | 1.3 | 1.3 | 1.2 | 1.2 | 1.2 | 1.1 | 1.1 | 1.0 | 0.9 |
| Ca | 0.9 | 0.9 | 0.9 | 0.9 | 0.8 | 0.8 | 0.8 | 0.7 | 0.6 |
| Fe | 2.1 | 2.1 | 2.1 | 2.0 | 2.0 | 1.9 | 1.8 | 1.7 | 1.5 |
| Mg | 0.2 | 0.2 | 0.2 | 0.2 | 0.2 | 0.2 | 0.2 | 0.2 | 0.2 |
| H | 2.6 | 2.6 | 2.6 | 2.6 | 2.6 | 2.6 | 2.6 | 2.6 | 2.6 |
| O | 59.5 | 59.1 | 58.6 | 57.6 | 56.6 | 55.6 | 53.7 | 51.7 | 47.8 |

In a first validation test, net peak areas calculated from simulated spectra and carbon content (in dry soil) by equation 19 are shown in Table 9. Comparison of calculated carbon content with model values demonstrated good agreement in all cases where the moisture correction factor (ie: utilizing the moisture calibration coefficients k1, k2 to derive the Equation (19) for determining the concentration of Carbon in the soil by measuring the net peak areas of the characteristic carbon and silicon peaks, and a measurement of the soil moisture content), was applied. However, it was found that the calculated carbon content values not corrected with moisture calibration coefficients, as per Equation (19), provides carbon content values that only agree with the model when carbon content is less than ˜3 wt % and moisture is less than 15%, while for higher values of carbon content and/or moisture, the calculated values (in the absence of moisture correction) and the modelled values do not agree.

TABLE 9
Net peaks area in spectra of wet homogenous soil with different moistures vs calculated
carbon content in dry soil (wt %) (error netSipa ± 0.4 cnt/1n, netCpa ± 0.2 cnt/1n).

| C wt % in dry soil | | | | | | | | |
|---|---|---|---|---|---|---|---|---|
| 0 | 1 | 2.5 | 5 | 7.5 | 10 | 15 | 20 | 30 |

Wet homogeneous soil with 5% Moisture

| | | | | | | | | | |
|---|---|---|---|---|---|---|---|---|---|
| netSipa, cnt/1n, ×10⁻⁶ | 65.2 | 64.3 | 63.6 | 61.7 | 60.1 | 58.9 | 54.7 | 52.2 | 45.6 |
| netCpa, cnt/1n, ×10⁻⁶ | 5.9 | 7.5 | 9.9 | 13.2 | 16.7 | 20.5 | 27.9 | 35.7 | 51.3 |
| Calculated C wt % in | 0.0 ± | 1.1 ± | 2.8 ± | 5.1 ± | 7.6 ± | 10.2 ± | 15.3 ± | 20.7 ± | 31.5 ± |
| dry mix with mo | 0.2 | 0.2 | 0.2 | 0.2 | 0.2 | 0.3 | 0.3 | 0.4 | 0.7 |
| correction ± Error | | | | | | | | | |
| Calculated C wt % in | 0.2 ± | 1.2 ± | 2.8 ± | 4.9 ± | 7.2 ± | 9.7 ± | 14.5 ± | 19.5 ± | 29.6 ± |
| dry mix w/o mo | 0.3 | 0.2 | 0.2 | 0.2 | 0.2 | 0.3 | 0.3 | 0.4 | 0.6 |
| correction ± Error | | | | | | | | | |

Wet homogeneous soil with 10% Moisture

| | | | | | | | | | |
|---|---|---|---|---|---|---|---|---|---|
| netSipa, cnt/1n, ×10⁻⁶ | 60.6 | 59.9 | 58.8 | 57.9 | 56.1 | 54.4 | 51.4 | 49.4 | 42.5 |
| netCpa, cnt/1n, ×10⁻⁶ | 5.9 | 7.3 | 9.2 | 12.7 | 15.9 | 19.4 | 26.7 | 33.9 | 48.4 |
| Calculated C wt % in | 0.3 ± | 1.3 ± | 2.7 ± | 5.2 ± | 7.6 ± | 10.2 ± | 15.5 ± | 20.7 ± | 31.4 ± |
| dry mix with mo | 0.2 | 0.2 | 0.2 | 0.2 | 0.2 | 0.3 | 0.4 | 0.5 | 0.7 |
| correction ± Error | | | | | | | | | |
| Calculated C wt % in | 0.5 ± | 1.4 ± | 2.6 ± | 4.9 ± | 6.9 ± | 9.2 ± | 13.9 ± | 18.5 ± | 28.0 ± |
| dry mix w/o mo | 0.1 | 0.2 | 0.2 | 0.2 | 0.2 | 0.2 | 0.3 | 0.4 | 0.6 |
| correction ± Error | | | | | | | | | |

Wet homogeneous soil with 15% Moisture

| | | | | | | | | | |
|---|---|---|---|---|---|---|---|---|---|
| netSipa, cnt/1n, ×10⁻⁶ | 56.2 | 55.5 | 55.2 | 54.1 | 52.4 | 51.1 | 48.1 | 45.9 | 40.0 |
| netCpa, cnt/1n, ×10⁻⁶ | 6.1 | 7.4 | 9.1 | 12.1 | 15.1 | 18.6 | 25.3 | 31.9 | 45.8 |
| Calculated C wt % in | 0.6 ± | 1.6 ± | 2.9 ± | 5.2 ± | 7.6 ± | 10.3 ± | 15.5 ± | 20.5 ± | 31.4 ± |
| dry mix with mo | 0.2 | 0.2 | 0.2 | 0.2 | 0.2 | 0.3 | 0.4 | 0.5 | 0.6 |
| correction ± Error | | | | | | | | | |
| Calculated C wt % in | 0.8 ± | 1.6 ± | 2.7 ± | 4.7 ± | 6.6 ± | 8.9 ± | 13.3 ± | 17.5 ± | 26.5 ± |
| dry mix w/o mo | 0.2 | 0.2 | 0.2 | 0.2 | 0.2 | 0.2 | 0.3 | 0.4 | 0.5 |
| correction ± Error | | | | | | | | | |

Wet homogeneous soil with 30% Moisture

| | | | | | | | | | |
|---|---|---|---|---|---|---|---|---|---|
| netSipa, cnt/1n, ×10⁻⁶ | 48.3 | 47.1 | 46.7 | 45.0 | 43.9 | 43.0 | 40.4 | 38.3 | 33.8 |
| netCpa, cnt/1n, ×10⁻⁶ | 5.5 | 6.6 | 8.0 | 11.2 | 13.8 | 16.5 | 21.8 | 27.6 | 39.3 |
| Calculated C wt % in | 0.3 ± | 1.4 ± | 2.7 ± | 5.6 ± | 8.0 ± | 10.4 ± | 15.3 ± | 20.6 ± | 31.1 ± |
| dry mix with mo | 0.2 | 0.2 | 0.2 | 0.2 | 0.2 | 0.3 | 0.4 | 0.4 | 0.6 |
| correction ± Error | | | | | | | | | |
| Calculated C wt % in | 0.8 ± | 1.6 ± | 2.5 ± | 4.6 ± | 6.3 ± | 8.0 ± | 11.5 ± | 15.2 ± | 22.8 ± |
| dry mix w/o mo | 0.2 | 0.2 | 0.2 | 0.2 | 0.2 | 0.2 | 0.3 | 0.3 | 0.5 |
| correction ± Error | | | | | | | | | |

In a second validation test, different amounts of water were added to a heterogeneous soil model. Again, the elemental content by depth in these wet, heterogenous soil models was calculated on the basis of data from Table 5 and equations similar to equations (13) and (14). The calculated data are shown in Table 10.

TABLE 10
The wet layered (heterogeneous) soil content with different moistures.

| Element | Soil depth (cm) | | | | | | | | | | | |
|---|---|---|---|---|---|---|---|---|---|---|---|---|
| symbol | 0-2 | 2-4 | 4-6 | 6-8 | 8-10 | 10-12 | 12-15 | \|15-20 | 20-30 | 30-40 | 40-50 | 50-60 |

Soil elemental content in wt % of soil with 5% moisture

| | | | | | | | | | | | | |
|---|---|---|---|---|---|---|---|---|---|---|---|---|
| C | 5.7 | 5.1 | 4.7 | 4.3 | 4.0 | 3.7 | 3.5 | 3.2 | 2.9 | 2.7 | 2.7 | 2.7 |
| Si | 32.6 | 32.3 | 32.1 | 31.8 | 31.5 | 31.2 | 30.9 | 30.3 | 29.4 | 28.3 | 27.4 | 26.7 |
| Na | 0.9 | 0.8 | 0.8 | 0.8 | 0.8 | 0.8 | 0.8 | 0.8 | 0.7 | 0.7 | 0.7 | 0.7 |
| Al | 5.8 | 5.8 | 5.7 | 5.7 | 5.7 | 5.7 | 5.6 | 5.6 | 5.5 | 5.4 | 5.3 | 5.2 |
| K | 1.5 | 1.5 | 1.5 | 1.4 | 1.4 | 1.4 | 1.4 | 1.4 | 1.3 | 1.3 | 1.2 | 1.2 |
| Ca | 1.1 | 1.1 | 1.1 | 1.1 | 1.1 | 1.1 | 1.1 | 1.1 | 1.1 | 1.0 | 9.0 | 9.0 |
| Fe | 2.5 | 2.5 | 2.5 | 2.5 | 2.5 | 2.5 | 2.5 | 2.5 | 2.5 | 2.5 | 2.5 | 2.5 |
| Mg | 0.3 | 0.3 | 0.3 | 0.4 | 0.4 | 0.4 | 0.4 | 0.5 | 0.6 | 0.7 | 0.8 | 0.9 |
| H | 0.5 | 0.5 | 0.5 | 0.5 | 0.5 | 0.5 | 0.5 | 0.5 | 0.5 | 0.5 | 0.5 | 0.5 |
| O | 49.2 | 50.1 | 50.8 | 51.5 | 52.1 | 52.7 | 53.3 | 54.1 | 55.4 | 56.8 | 49.8 | 50.6 |

Soil elemental content in wt % of soil with 10% moisture

| | | | | | | | | | | | | |
|---|---|---|---|---|---|---|---|---|---|---|---|---|
| C | 5.5 | 4.9 | 4.5 | 4.1 | 3.8 | 3.6 | 3.3 | 3.1 | 2.8 | 2.6 | 2.6 | 2.5 |
| Si | 31.1 | 30.9 | 30.6 | 30.3 | 30.1 | 29.8 | 29.5 | 28.9 | 28.0 | 27.0 | 26.2 | 25.5 |
| Na | 0.8 | 0.8 | 0.8 | 0.8 | 0.8 | 0.8 | 0.8 | 0.7 | 0.7 | 0.7 | 0.7 | 0.6 |
| Al | 5.5 | 5.5 | 5.5 | 5.5 | 5.4 | 5.4 | 5.4 | 5.3 | 5.2 | 5.1 | 5.1 | 5.0 |
| K | 1.4 | 1.4 | 1.4 | 1.4 | 1.4 | 1.3 | 1.3 | 1.3 | 1.3 | 1.2 | 1.2 | 1.2 |
| Ca | 1.0 | 1.0 | 1.0 | 1.0 | 1.0 | 1.0 | 1.0 | 1.0 | 1.0 | 1.0 | 8.6 | 8.6 |
| Fe | 2.4 | 2.4 | 2.4 | 2.4 | 2.4 | 2.4 | 2.4 | 2.4 | 2.4 | 2.4 | 2.4 | 2.4 |
| Mg | 0.3 | 0.3 | 0.3 | 0.3 | 0.4 | 0.4 | 0.4 | 0.5 | 0.6 | 0.7 | 0.8 | 0.9 |
| H | 1.0 | 1.0 | 1.0 | 1.0 | 1.0 | 1.0 | 1.0 | 1.0 | 1.0 | 1.0 | 1.0 | 1.0 |
| O | 51.0 | 51.8 | 52.6 | 53.2 | 53.8 | 54.3 | 54.9 | 55.7 | 57.0 | 58.3 | 51.6 | 52.3 |

Soil elemental content in wt % of soil with 15% moisture

| | | | | | | | | | | | | |
|---|---|---|---|---|---|---|---|---|---|---|---|---|
| C | 5.2 | 4.7 | 4.3 | 3.9 | 3.6 | 3.4 | 3.2 | 2.9 | 2.6 | 2.5 | 2.4 | 2.4 |
| Si | 29.8 | 29.5 | 29.3 | 29.0 | 28.8 | 28.5 | 28.2 | 27.7 | 26.8 | 25.8 | 25.0 | 24.4 |
| Na | 0.8 | 0.8 | 0.8 | 0.8 | 0.7 | 0.7 | 0.7 | 0.7 | 0.7 | 0.7 | 0.6 | 0.6 |
| Al | 5.3 | 5.3 | 5.2 | 5.2 | 5.2 | 5.2 | 5.1 | 5.1 | 5.0 | 4.9 | 4.8 | 4.8 |
| K | 1.4 | 1.4 | 1.3 | 1.3 | 1.3 | 1.3 | 1.3 | 1.2 | 1.2 | 1.2 | 1.1 | 1.1 |
| Ca | 1.0 | 1.0 | 1.0 | 1.0 | 1.0 | 1.0 | 1.0 | 1.0 | 1.0 | 0.9 | 8.2 | 8.2 |
| Fe | 2.3 | 2.3 | 2.3 | 2.3 | 2.3 | 2.3 | 2.3 | 2.3 | 2.3 | 2.3 | 2.3 | 2.3 |
| Mg | 0.2 | 0.3 | 0.3 | 0.3 | 0.4 | 0.4 | 0.4 | 0.5 | 0.5 | 0.7 | 0.8 | 0.8 |
| H | 1.4 | 1.4 | 1.4 | 1.4 | 1.4 | 1.4 | 1.4 | 1.4 | 1.4 | 1.4 | 1.4 | 1.4 |
| O | 52.7 | 53.4 | 54.1 | 54.8 | 55.3 | 55.8 | 56.4 | 57.2 | 58.4 | 59.6 | 53.2 | 53.9 |

Soil elemental content in wt % of soil with 30% moisture

| | | | | | | | | | | | | |
|---|---|---|---|---|---|---|---|---|---|---|---|---|
| C | 4.6 | 4.2 | 3.8 | 3.5 | 3.2 | 3.0 | 2.8 | 2.6 | 2.3 | 2.2 | 2.2 | 2.1 |
| Si | 26.3 | 26.1 | 25.9 | 25.7 | 25.4 | 25.2 | 24.9 | 24.5 | 23.7 | 22.9 | 22.1 | 21.6 |
| Na | 0.7 | 0.7 | 0.7 | 0.7 | 0.7 | 0.7 | 0.6 | 0.6 | 0.6 | 0.6 | 0.6 | 0.5 |
| Al | 4.7 | 4.7 | 4.6 | 4.6 | 4.6 | 4.6 | 4.5 | 4.5 | 4.4 | 4.3 | 4.3 | 4.2 |
| K | 1.2 | 1.2 | 1.2 | 1.2 | 1.1 | 1.1 | 1.1 | 1.1 | 1.1 | 1.0 | 1.0 | 1.0 |
| Ca | 0.9 | 0.9 | 0.9 | 0.9 | 0.9 | 0.9 | 0.9 | 0.9 | 0.9 | 0.8 | 7.2 | 7.3 |
| Fe | 2.0 | 2.0 | 2.0 | 2.0 | 2.0 | 2.0 | 2.0 | 2.0 | 2.0 | 2.0 | 2.0 | 2.0 |
| Mg | 0.2 | 0.2 | 0.3 | 0.3 | 0.3 | 0.3 | 0.4 | 0.4 | 0.5 | 0.6 | 0.7 | 0.7 |
| H | 2.6 | 2.6 | 2.6 | 2.6 | 2.6 | 2.6 | 2.6 | 2.6 | 2.6 | 2.6 | 2.6 | 2.6 |
| O | 56.9 | 57.5 | 58.1 | 58.7 | 59.2 | 59.6 | 60.1 | 60.8 | 61.9 | 63.0 | 57.3 | 57.9 |

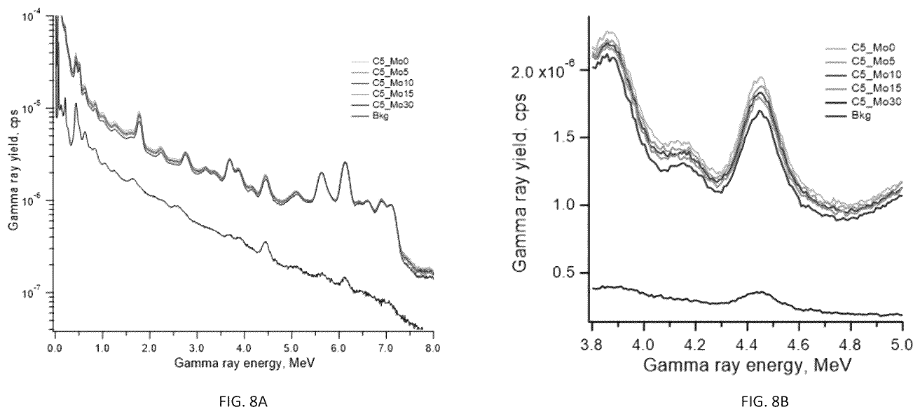

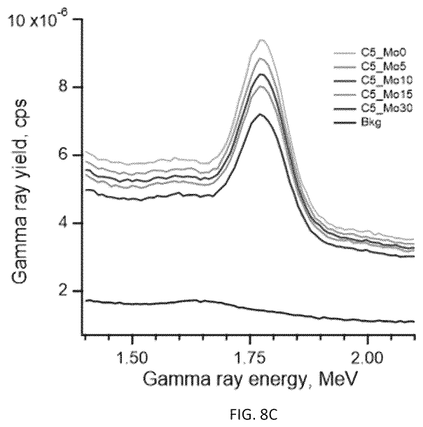

The simulated spectra of layered soil, in the upper 10 cm layer of wet soil (based on the average elemental content in the upper 10 cm of the soil, based on the values of the elemental content in the different soil layers provided in Table 10 above), and with different moistures, are shown in FIGS. 8A to 8C.

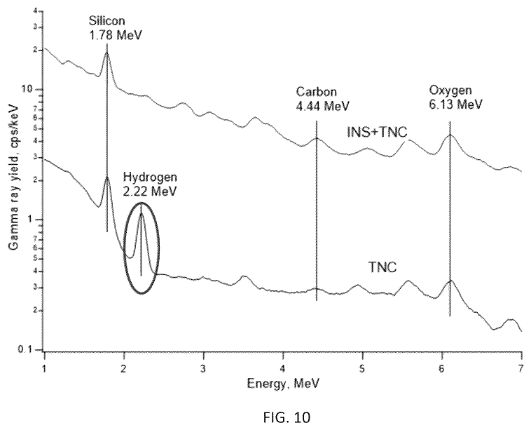

FIG. 10A is a gamma spectra of the full energy range, with the simulated wet sand-carbon calibration pits (with 10% carbon by weight at 0% moisture) for different moisture levels, ranging from 0% moisture by weight to 30% moisture by weight. FIG. 5B is a close-up view 10 of the gamma spectra of FIG. 5A, showing the peak characteristic of Carbon, and FIG. 5C is a close-up view of the gamma spectra of FIG. 5A, showing the peak characteristic of Silicon. As may be appreciated from FIGS. 5A to 5C, the peak area of the characteristic Carbon peaks (i.e. the peak with a centroid at 4.44 MeV) slightly decreased as moisture content increased, while the peak area of the characteristic Silicon peaks (i.e. the peak with a centroid at 1.78 MeV) also decreased as the moisture content increased. As the relative amounts of both carbon and silicon decrease with increased moisture levels, as reflected in Table 6, the simulated gamma spectra approximately correspond with the values shown in Table 6.

Net peak area calculation results and average carbon content in the upper 10 cm of dry soil (from simulated spectra and equation 19) are shown in Table 11. Average carbon content in the upper 10 cm from calculations using calibrations coefficients for dry sand-carbon mixtures are also presented in Table 11. As can be seen, soil carbon content determined by the moisture corrected formula agrees with the modeled carbon content (5 Cwt % in the upper 10 cm layer), while carbon content calculated with calibration coefficients without moisture correction declined as compared to the modeled values.

TABLE 11
Net peak areas in spectra of layered soil (5 wt % carbon content in upper
10 cm layer in dry soil) with different moistures, and calculated soil
carbon content in the upper 10 cm soil layer with and without moisture
correction (error: netSipa ±0.5 cnt/1n, netCpa ±0.2 cnt/1n).

| Moisture, % | 0 | 5 | 10 | 15 | 30 |
|---|---|---|---|---|---|
| netSipa, cnt/1n, ×10⁻⁶ | 66.5 | 61.1 | 56.7 | 53.4 | 45.5 |
| netCpa, cnt/1n, ×10⁻⁶ | 13.5 | 13.1 | 12.4 | 11.8 | 10.6 |
| C wt % calculated | 4.9 ± 0.2 | 5.1 ± 0.2 | 5.1 ± 0.2 | 5.1 ± 0.2 | 5.1 ± 0.2 |
| with mo correction | | | | | |
| C wt % calculated | 4.9 ± 0.2 | 4.9 ± 0.2 | 4.7 ± 0.2 | 4.5 ± 0.2 | 4.2 ± 0.2 |
| without mo correction | | | | | |

Thus, the discussed examples indicate that moisture correction should be applied when calculating soil carbon content from the neutron stimulated gamma spectra of wet soil, to obtain a more accurate determination of the elemental content of the soil. Although the examples presented in this present disclosure are based on determining the elemental content of carbon, it will be appreciated that the same methods may be used to obtain more accurate measurement of the content of other elements in the soil which are measured from inelastic neutron scattering spectra. Such elements include, but are not limited to: Silicon, Oxygen, Iron and Aluminum.

The determined corrections of calibration coefficients based on Monte-Carlo computer simulations described above may be utilized in real world gamma spectra obtained from soil. Accepting that real-world dependencies of calibration coefficients for carbon or other elements of interest, based on varying moisture levels, correspond to the simulated dependencies of calibration coefficients with varying moisture levels, it will be appreciated that the calculated dependencies provided in equations (17) and (18) (using carbon as an example) may be used in field soil measurements. However, these calculated dependencies should be normalized with the value of coefficients at mo=0; primarily, equations (17) and (18) should be multiplied by the ratio of k1ₑₓₚ (0)/k1_{calc} (0) and k2ₑₓₚ (0)/k2_{calc} (0). This normalization adjustment provides the following equations for obtaining the real-world dependencies of calibration coefficients with varying moisture levels, as follows:

$$k {1}_{exp} ( m o ) = ( 1 + 0 \text{.00592} \cdot mo + 0.0000414 \cdot {mo}^{2} ) \cdot k {1}_{exp} ( 0 ) \qquad ( 20 )$$

$$k {2}_{exp} ( m o ) = ( 1 - 0.01 \; 224 \cdot mo + 0.0000 \; 965 \cdot {mo}^{2} ) \cdot k {2}_{exp} ( 0 ) \qquad ( 21 )$$

where the k1ₑₓₚ (0) and k2ₑₓₚ (0) are normalized calibration coefficients for carbon obtained from real world gamma spectra acquired from calibration scans performed on dry sand-carbon calibration pits, the calibration pits having known amounts of carbon and silicon and substantially zero moisture levels. The preparation of dry sand-carbon calibration pits may be prepared in accordance with the procedures described herein for the preparation of sand-polyethylene powder pits.

Briefly, the sand-carbon calibration pits would be prepared to contain varying, known amounts of carbon and silicon in each calibration block, by determining the density of the sand and the coconut shell components to be mixed together to form a homogenous mixture, with the coconut shell providing the known amounts of carbon and the sand providing the known amounts of silicon in the calibration pit. Prior to measuring and mixing together the sand and the coconut shell, each component is tested with a moisture measuring instrument to confirm there is less than 5% moisture in each component; if excess moisture is found, the sand or the coconut shell (as the case may be) can be dried through heating at a low temperature and/or by aeration, until the component contains less than 5% moisture by weight. The calibration pits may be configured, for example, to contain carbon in the amounts of 0%, 5%, 10%, 15%, 20%, 25% and 30% carbon by weight, respectively. The homogenous mixture may also be tested with a moisture reading instrument, to confirm that the homogenous mixture of the calibration pit contains less than 5% moisture, which is a typical amount of moisture in sand that is dry. If the moisture content of the mixture is greater than 5%, the mixture may be aerated to evaporate the excess moisture.

### Measuring Soil Moisture Content

In another aspect of the present disclosure, methods and systems are provided for utilizing pulsed fast thermal neutron gamma analysis (PFTNA) for soil moisture measurement. In some embodiments, a PFTNA system acquires the spectra of gamma rays, which are radiated from the soil when the soil is exposed to neutron irradiation. These acquired spectra contain gamma peaks due to characteristic gamma lines from the elements of the soil.

Hydrogen nuclei have a clear gamma peak in the TNC spectra at 2.223 MeV due to a thermal neutron reaction. The area of this gamma peak at 2.223 MeV depends primarily on the amount of water in the soil. Thus, the PFTNA method and apparatus may be utilized to determine the water content in the soil. Advantageously, the PFTNA method of determining soil moisture content is a non-destructive, in-situ method that may be applied in a continuous scanning mode, thereby allowing for the measurement of varying soil moisture content across a large area, such as a field comprising many acres.

### PFTNA System

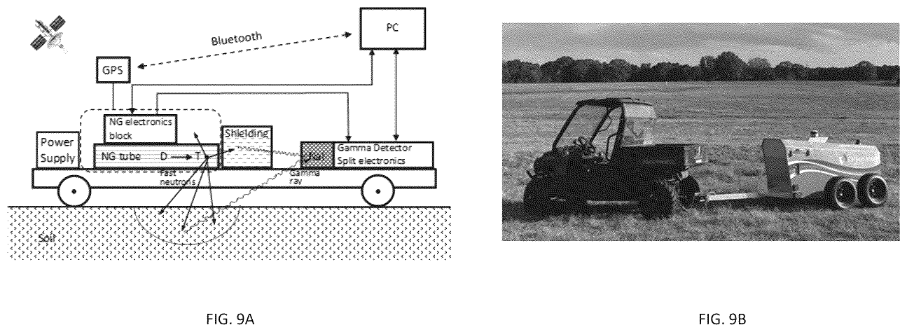

A mobile PFTNA system for acquiring gamma spectra and measuring moisture content includes a pulsed neutron generator, a scintillation detector for detecting the gamma rays reflected from the soil, a power source and electronics for operating the system. A pulsed neutron generator is used as a neutron source; an example of a pulsed neutron generator that may be used for this purpose is the model MP 320 portable neutron generator manufactured by Thermo Fisher Scientific. The gamma rays are registered by scintillation detectors; in an example embodiment of the apparatus, three large-volume sodium iodide (Nal) crystal scintillator detectors (having a total volume of approximately 7.5 L) may be used, such as Nal gamma detectors manufactured by Scionix. One example of a detector assembly comprises an Nal crystal coupled to a photomultiplier tube (PMT). The Nal detector assemblies may be provided with corresponding electronics, such as manufactured by XIA LLC. A power system for powering the apparatus may comprise, for example, four 12V batteries, an DC-AC inverter, and a charger. Optionally, the apparatus may be provided with a GPS device, which provides geographical coordinates of the system during scanning operations. The system is operated by a laptop. A schematic drawing of the system and a photograph of an example of the system mounted to a mobile cart pulled by a tractor, are shown in FIGS. 9A and 9B, respectively.

The system is configured to acquire two gamma spectra from the soil simultaneously. The first gamma spectrum is obtained when the pulsed neutron generator emits a pulse, which causes gamma rays to be emitted from the soil under irradiation, primarily due to inelastic neutron scattering (INS). The second gamma spectrum is acquired in-between the pulses emitted by the pulsed neutron generator, with the resulting gamma spectra primarily due to thermal neutron capture (TNC). In general, the INS spectrum contains characteristic gamma peaks for elements including Carbon, Silicon and Oxygen, which appear due to an INS reaction (FIG. 2). Whereas the TNC spectrum contains a characteristic gamma peak of the Hydrogen nuclei at 2.223 MeV (amongst other gamma peaks); see for example the spectrum shown in FIG. 10. The INS and TNC gamma spectra peaks may be analyzed to determine the elemental content of the soil; and in particular, the characteristic Hydrogen peak in the TNC gamma spectra may be utilized to measure the moisture content in the soil. FIG. 10 is an example of both a TNC gamma spectra and a combined INS and TNC gamma spectra taken of the same soil sample. It may be appreciated that the Hydrogen peak (having a centroid at 2.22 MeV) is more prominent in the TNC spectra. The area of the hydrogen peak shown in the TNC spectra is proportionate to the amount of water content in the soil, as the Applicant has found that water is the primary component in the soil that contains hydrogen atoms.

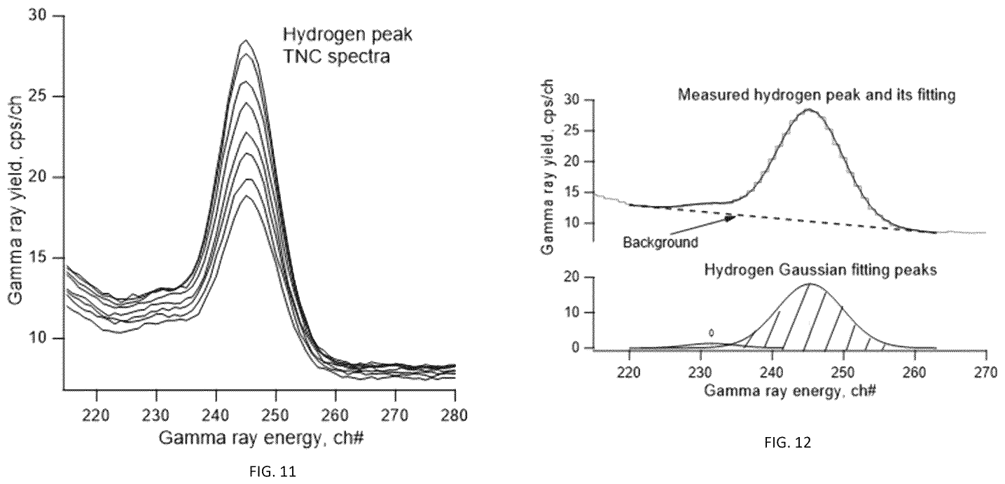

A portion of the gamma spectra, containing the characteristic hydrogen peak at 2.22 MeV, is shown for several TNC gamma spectra acquired from different soil sites in FIG. 11. The area of the hydrogen peak is proportionate to the amount of hydrogen (or in other words, the amount of water) contained in the soil. The hydrogen peak area may be calculated by Gaussian fitting of this peak, shown for example in FIG. 12. As viewed in FIG. 12, the first graph showing square peaks represents the residual measurements, or in other words, the difference between the measured gamma spectra and the fitting of the data by channels. In the second graph, the black curves represent the fitting Gaussian curves, and the broken line represents the linear background gamma rays. In the third graph, the shaded peak represents the gamma spectra obtained only from the TNC reactions with the hydrogen atoms, thereby representing a corrected curve that corresponds to the amount of water in the soil being measured.

The increasing water content in soil (otherwise referred to herein as soil moisture or soil moisture content) increases the peak area of the hydrogen peak at 2.22 MeV when the soil is irradiated by fast neutrons. The hydrogen nuclei are effective moderators for neutrons due to the hydrogen atom containing a neutron and a proton that are approximately equal in mass. Thus, the intensity of the characteristic hydrogen gamma line (peak, having a centroid at 2.223 MeV), may increase superlinearly with increasing the hydrogen content in soil.

### Method for Hydrogen Peak Measurements and the Determination of Soil Moisture

To obtain the gamma spectrometry data, the PFTNA system may be operated in a scanning mode or a static mode on the field or site of interest. In static mode, the PFTNA system is positioned at a place of interest for acquiring the INS and TNC spectra. The PFTNA system (neutron flux of ˜10⁷ neutrons per second, three Nal gamma detectors having a volume of 2.4 dm³ each, for a total volume of approximately 7.2 dm³) may be operated for approximately 30 seconds to obtain a TNC spectrum having a peak with a centroid at 2.223 MeV (referred to herein as the “hydrogen peak”). The Applicant has found that, comparing the calculated hydrogen content based on the area of the hydrogen peak to the measurement of the soil moisture obtained by other methods such as using instrumentation, may yield an estimate for the amount of water content having a statistical error within 1-2% as compared to other methods for measuring the water content of the same soil sample. Advantageously, the Applicant has found that obtaining measurements of the water content with a spectra acquisition time of approximately 30 seconds for the soil sample yields adequate information for water content determination, unlike the measurement of other elements utilizing the TNC and INS spectra; for example, by comparison, the acquisition of gamma spectra for determining carbon content in soil may require acquisition time of approximately 15 minutes.

When a field is surveyed for elemental content by operating the PFTNA system in scanning mode the PFTNA system may be moving across the field at a speed of approximately 5 to 6 km/h. The acquired gamma spectra may be saved each 30 seconds (or other selected time interval), together with the geographical coordinates provided, for example, by a GPS component. Each saved segment of acquired gamma spectra will have a start time and an end time for the acquisition time, and the GPS coordinate assigned to that saved segment will be the midpoint location between the starting location and the end location where the PFTNA system was located on the field during the time in which the gamma spectra segment was acquired. Thus, the system processes the acquired spectra of the saved segment to provide an average of the spectra acquired at and between the two neighboring points, and assigns the averaged spectra to a location that is located midway between the two neighboring points. The geographical coordinates of the midpoint between two neighboring points of the acquired and saved gamma spectra is calculated, for example by measuring the location of the midpoint between the coordinates of each location of the two neighboring points as determined, for example, by the GPS component. The hydrogen peak area, discussed herein, is thus calculated from the midpoint TNC spectra. Using a calibration equation (as described herein and shown, for example, in FIG. 16), the moisture content may be determined and attributed to the midpoint location calculated between the two neighboring locations where the acquisition of the gamma spectra began and ended. The data set of midpoint coordinates and values of moisture calculated at each of these midpoints may be used for generating a moisture distribution map over the surveyed field.

One approach to obtain spectra at points evenly distributed across a field of interest is to firstly divide the field into a number of sections, each section having approximately the same surface area, and moving the system across each section for approximately the same amount of time on each section. Another approach is to plan and implement a travel trajectory that evenly covers the field, using the GPS to track where the system has travelled across the field, and to correlate the saved gamma spectra acquired by the PFTNA system with a geographic location on the field, as determined for example by the GPS device. The GPS device may be integrated into the PFTNA system, or it may be a separate component that is in communication with the processor of the PFTNA system.

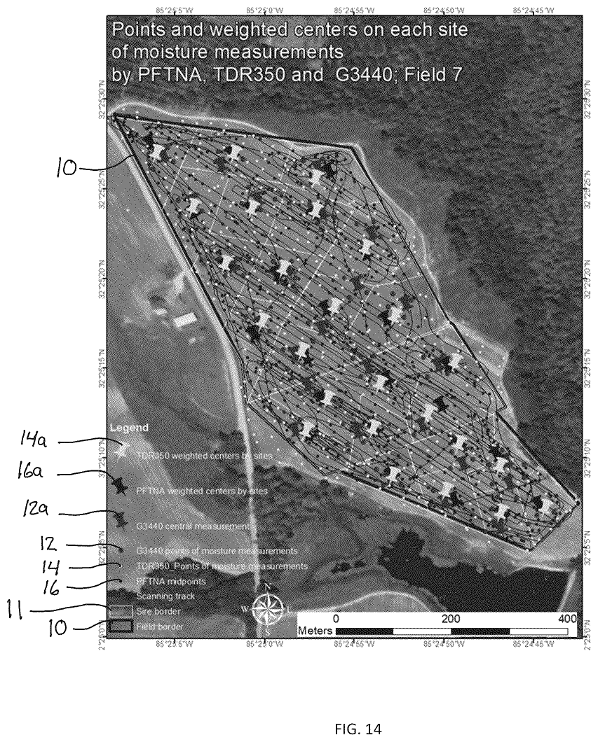

In a validation study, if soil moisture measurement was made by some other instrument like Gauge 3440 Troxler™ Surface Moisture-Density Gauge using by nuclear method or TDR 350 using the time domain reflectometry method, the points of measurements obtained from instruments and from PFTNA measurements may be sorted by sites, and an average weighted value and its position may thus be determined. Thus, the soil moisture measurements obtained using different methods may then be compared, and due to the weighted centers of data obtained from one site, the geographic locations associated with each moisture measurement data point may be disposed very close each other. For example, see FIG. 14, which illustrates the locations of moisture measurements taken across a geographic area 10, shown as a black border on the map of FIG. 14. The geographic area 10 is divided into a plurality of smaller sites 11, shown with white borders on the map of FIG. 14. In this example data set, the individual measurement locations 12 taken by the G3440 Troxler™ gauge are illustrated by grey dots, and the weighted centers 12a for all the moisture measurements taken by the G3440 Troxler™ gauge within a given site 11, are illustrated as grey thumbtacks. Likewise, the individual measurement locations 14 and the weighted centers 14a for all the individual measurements within a given site 11, for moisture measurements taken by the TDR 350 gauge, are illustrated as white dots and white thumbtacks, respectively. For the moisture measurements determined from gamma spectra obtained by the mobile PFTNA system, the midpoints between the starting and ending locations where the mobile cart was located when the gamma spectrum acquisition began and ended, are indicated by the black dots 16 on the map, and the weighted centers for all the midpoint locations within a given site 11 are indicated by black thumbtacks 16a on the map. As viewed in FIG. 14, the grey, white and black thumbtacks, representing the weighted centers for all the respective measurements of each type within a given site, are located close to one another in each site 11. Therefore, the moisture content measurements, as obtained by each of the three different methods described above taken on the same date (with the PFTNA system, the G3440 Troxler™ gauge and the TDR 350 gauge), should be relatively close to one another, given the close physical proximity of the weighted centers for each of these different data sets.

### Hydrogen Peak Background Measurement

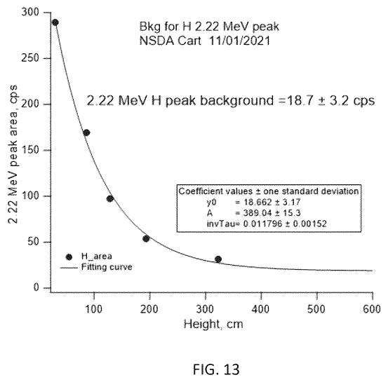

The gamma spectra obtained from the PFTNA system consists of two parts: namely, the spectra obtained from the soil sample under analysis, and the background spectra obtained from the equipment and materials of the PFTNA system itself under neutron irradiation. For the element of hydrogen, the main source of the hydrogen peak in the background spectra is neutron shielding materials present on the PFTNA system, such as compressed boric acid. To take into account the contribution of hydrogen present in the PFTNA equipment itself to the hydrogen peak, the system's background spectra is also obtained. To obtain the background spectra, the PFTNA system is raised to a position three to five meters above the ground, and then operated for 30 seconds to obtain the gamma spectra. As may be viewed in FIG. 13, the area of the hydrogen peak measured from gamma spectra acquired with the PFTNA positioned above the ground at different heights plateaus and reaches a steady state at a height of more than 300 cm off the ground. This background measurement of the hydrogen peak may be subsequently used in both the calibration measurements and in the determination of hydrogen content in soil samples.

### Calibration of the PFTNA System

The PFTNA system may be calibrated by preparing a set of calibration blocks, each block containing a homogenous mixture of known amounts of sand and polyethylene powder. When preparing the calibration blocks, the moisture content of the sand and the polyethylene powder are measured, such as with a nuclear gauge (for example the 3440Plus gauge manufactured by Troxler), to confirm the moisture content is less than 5%. For example, not intended to be limiting, seven calibration pits may be prepared with a mixture ratio for a plurality of carbon-containing pits, the pits having 2.5%, 5%, 10% and 15%, 20%, 30% and 40% carbon by weight, are calculated, as well as having known amounts of hydrogen from the polyethylene powder in known amounts, for example, containing the equivalent amount of hydrogen as would be contained in a soil sample having 3.8%, 7.5%, 15%, 22.5%, 30%, 45% and 60% moisture content by weight, respectively. The calibration blocks are prepared by taking into account the moisture content of the sand and the polyethylene powder as measured by a gauge, with the amounts of sand and polyethylene powder calculated to provide a calibration block mixture with known amounts of hydrogen, carbon and silicon. The amount of hydrogen in the calibration block is provided by the polyethylene powder, and a negligible contribution by any residual moisture that is present in the dry sand and/or the polyethylene powder. If the moisture content of either the sand or the polyethylene powder is measured to exceed 5%, then the sand and/or polyethylene powder may firstly be aerated to evaporate the moisture, before preparing the calibration blocks. Advantageously, using polyethylene powder to provide the hydrogen content of the calibration blocks provides for a stable and known amount of hydrogen, and avoids the possibility of the hydrogen content of the calibration blocks fluctuating during the calibration procedure.

To calibrate the PFTNA system, the moisture of the calibration blocks are measured with a nuclear gauge to confirm the moisture content is less than 5%. Then, the PFTNA system is positioned over the 0% carbon calibration block and the gamma spectra is acquired over a set time interval; for example, 45 minutes. Once the spectra is acquired, the PFTNA system is relocated to the 2.5% carbon calibration block and the gamma spectra is acquired over the same time interval (eg: 45 minutes). This process is repeated to acquire gamma spectra for each of the calibration blocks (for example, containing 5%, 10%, 15%, 20%, 30% and 40% carbon, for example).

The background peak calibration is performed by positioning the PFTNA system at ground level in an open area (away from the calibration blocks) and the gamma spectra is acquired over the set time interval. This process is repeated after elevating the PFTNA system above the ground at different heights; for example, at 0.5 meters, 1.0 meters, 1.5 meters, 2.0 meters and 3.0 meters above the ground (such as by suspending the PFTNA system above the ground with a crane). Additionally, the bulk density of each calibration block is measured, for example, by using a nuclear gauge inserted at a depth of 100 mm from the surface of the calibration block. Calculations are then performed on the acquired gamma spectra to obtain the calibration coefficients for obtaining the net peak areas of hydrogen, carbon and silicon, ad described elsewhere in this disclosure. The hydrogen calibration coefficients, as determined for the PFTNA system during calibration, are then used to obtain net hydrogen peak area measurements and to calculate the moisture content of a soil, as measured by that PFTNA system.

Validation Study—Moisture Content Measurement using PFTNA System

Several agricultural fields in the state of Alabama (of the United States of America) were previously surveyed using a PFTNA system to determine soil elemental content (mainly carbon) over several years. At the same time, soil moisture data was obtained from the same fields using sampling and weight analysis and measurements by instruments (TDR 350, G3440). The acquired gamma spectra data from these studies included data regarding hydrogen peak area in the acquired TNC gamma spectra. The fields were divided into sections, and the acquired data (hydrogen peak area and moisture data) was sorted by each section on the field.

FIG. 14 illustrates one of the fields for which data was acquired; the map of FIG. 14 (previously discussed above) shows the path of the PFTNA system during the gamma spectra acquisition operations, whereas the black points on this line show the midpoints between two neighboring spectra that were saved to the system. These midpoints were sorted by site, the position of weighted center of these midpoints attributed to site 11 (the weighted centers represented in FIG. 14 by black thumbtacks) were determined, the average value of hydrogen peak area was found and attributed to this weighted center. The positions of the grey, white and black thumbtacks 12a, 14a and 16a on each site11, marked the location of the weighted center, within each site 11, of the individual moisture measurements obtained by different methods (ie: as obtained from the TDR350 gauge, the G3440 gauge and PFTNA acquired spectra, respectively). As viewed in FIG. 14, the locations of these moisture measurements, taken by different methods, are in close proximity to one another, and thus the moisture of the soil as obtained by these three different methods may be compared. The instrumental corrections of moisture meters and value of background at the hydrogen peak determination were taken into account.

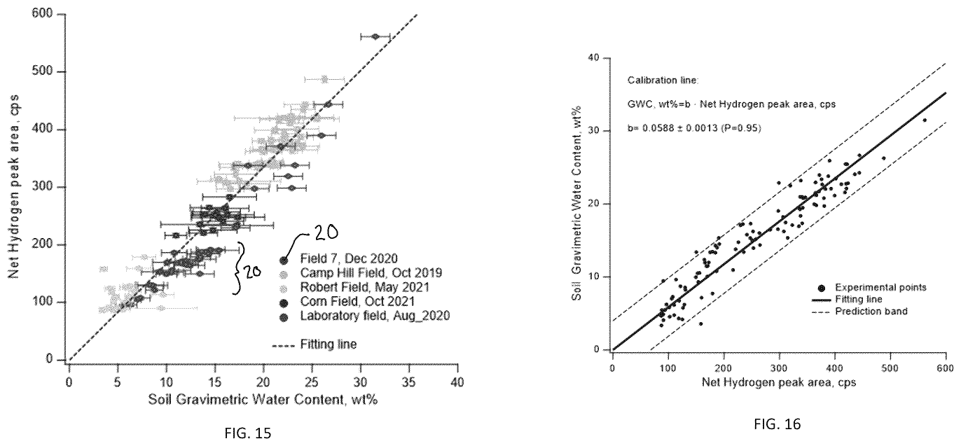

The values of Net Hydrogen peak area versus gravimetric water content defined by the hydrogen peak area, TDR 350 or G3440 on different fields, including the data points 20 from the Field 7 survey illustrated in the map of FIG. 14, are shown in FIG. 15. As illustrated in this plot, the variance in soil moisture determination is relatively large, ranging between approximately 2% to 4% weight; however, this variance likely reflects actual soil moisture variation between the locations where the measurements were taken, at a distance between 10 cm and 500 cm from one another. Due to the soil analyzed volume by weight analysis, the sample volumes were in the range of 1 to 2 dm³ of soil (for samples measured by TDR-350 and G3440). On the other hand, advantageously, the PFTNA system acquires neutron stimulated gamma response from a soil volume in approximately the range of 200 to 300 dm³, multiplied by 40 meters of travel of the PFTNA system during a 30 second time interval, yielding a total soil volume measured by a given gamma spectra to be approximately 8 m³. Thus, the hydrogen peak area obtained from the resulting gamma spectra represents the average soil moisture of this volume of soil, which results in a lower error in the soil moisture measurements as compared to measuring discrete, small samples spaced apart from one another, which discrete soil moisture measurements reflect a true variation in the soil moisture as between the different discrete soil sample measurements. A further advantage of the soil moisture determination obtained by scanning the field with a mobile PFTNA system is relatively fast and less labor intensive, as compared to measuring samples and conducting a weight analysis and moisture measurements of those soil samples utilizing the TDR 350 or the G3440 instruments, which is more labor intensive and time consuming. A further advantage of the soil moisture determination obtained by scanning a field with a mobile PFTNA system, is that the system may be configured to determine both volumetric and gravimetric water content without having to obtain the corresponding soil bulk density of each sample.

As may be appreciated from the plot shown in FIG. 15, comparing the net hydrogen peak area of the acquired gamma spectra and the soil gravimetric water content values as determined by instrumentation (as measured by the time domain reflectometry method utilizing a TDR-350 gauge and the nuclear method utilizing the G3440 gauge, as would be known to a person skilled in the art), and taking into consideration the anticipated soil moisture variation between discrete samples, there exists a linear correlation between the net hydrogen peak areas obtained from the gamma spectra and the soil gravimetric water content. Thus, a calibration line for calculation of the gravimetric water content, based on the measured hydrogen peak areas, is obtained as shown in FIG. 16. Similar calibration lines for calculating volumetric water content, based on the measured hydrogen peak areas, may be established with the same confidence level of error.

In this study, the error of regression coefficient was equal to 0.0013 at 95% confidence level, with the calibration line passing through zero on both axes of the plot. The calibration line, as obtained in the plot of FIG. 16, provides the moisture calibration equation that is used to determine the soil moisture content of the soil, based on the net hydrogen peak areas obtained from TNC gamma spectra acquired from the field by the mobile PFTNA system. For example, as shown in FIG. 16, the Gravimetric Water Content (GWC), wt % may be calculated as follows:

$$GWC , wt \% = b ( Net Hydrogen Peak Area , cps ) \qquad ( 22 )$$

where b=0.0588+/−0.0013.

The Applicant determined in this study that the error of moisture determination, obtained from the acquired gamma spectra, did not exceed +/−0.4% wt. as compared to the discrete soil moisture measurements obtained from the gauges. Thus, the measurement of soil gravimetric water content utilizing a mobile PFTNA system, as described herein, provides a faster method of measuring and mapping the soil moisture content across an entire field as compared to other methods utilizing water content measurements of discrete soil samples. For example, the acquisition of gamma spectra using a mobile PFTNA system to analyze the gravimetric (or volumetric) water content of the soil across a 20 ha field may be completed in approximately one hour. However, if the elemental composition of the soil is to be determined at the same time, the Applicant has found that the time intervals for obtaining gamma spectra to analyze, for example, the carbon content of a soil, such that it may take approximately five to eight hours to scan a 20 ha field. Furthermore, as demonstrated herein, utilizing a mobile PFTNA system to measure soil gravimetric water content yields relatively accurate data that sufficiently corresponds to soil gravimetric water content analysis obtained by other methods. Likewise, a mobile PFTNA system may be configured to measure soil volumetric water content yields to provide the same accuracy data that sufficiently corresponds to soil volumetric water content analysis obtained by other methods.

Although Equation (22), above, is obtained from experimental data sets as described herein, it will be appreciated that a calibration procedure may be performed on a PFTNA system, using calibration blocks containing known amounts of carbon and hydrogen (from a mixture of dry sand and dry polyethylene powder), and acquiring TNC gamma spectra of each calibration block, as described elsewhere in this disclosure. Net hydrogen peak areas are calculated from the acquired TNC spectra, and a plot, similar to the plot shown in FIG. 16, may be prepared to illustrate the relationship between the net hydrogen peak areas obtained from the TNC spectra and the moisture content of each calibration block (wherein the known amount of hydrogen, in each calibration block, is converted to represent an equivalent of the moisture content in each calibration block). The calibration line obtained from such a calibration procedure, may then be used as the moisture calibration equation for determining moisture content of soil surveyed by that mobile PFTNA system, derived from TNC gamma spectra acquired by the mobile PFTNA system.

### Soil Moisture Mapping

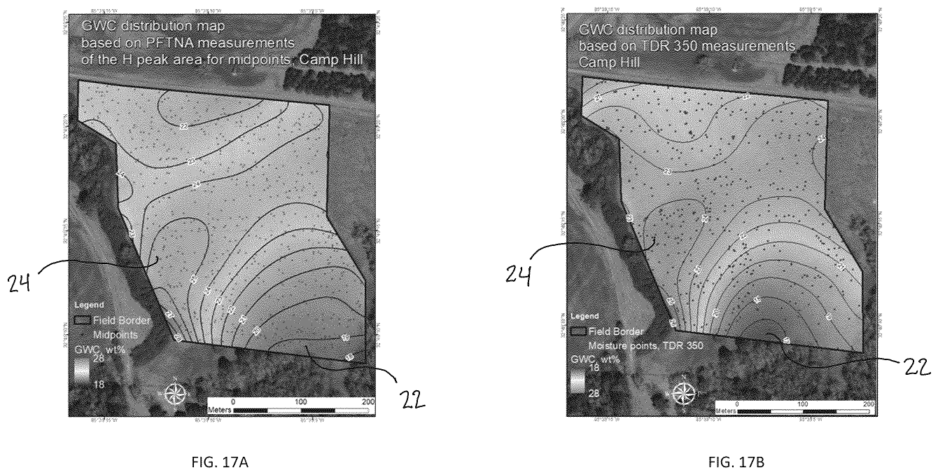

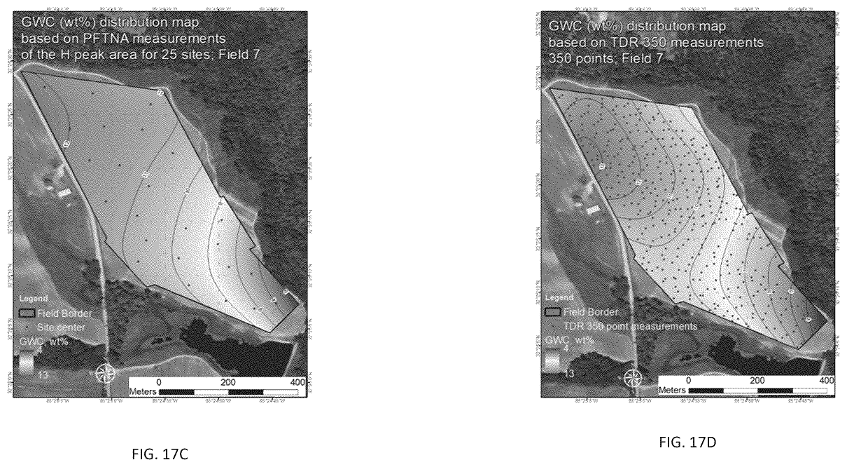

In another aspect of the present disclosure, the data acquired from a mobile PFTNA system for measuring gravimetric water content of a soil may be utilized to construct a soil moisture distribution map. Examples of soil moisture distribution maps are shown in FIGS. 17A to 17D, constructed from the data acquired during the validation studies discussed herein. FIGS. 17A and 17B show soil moisture distribution maps for a field referred to as “Camp Hill”, with the map of FIG. 17A constructed from gamma spectra acquired from a mobile PFTNA system, and the map of FIG. 17B constructed from TDR 350 soil moisture measurements taken on the same day as the PFTNA scan was conducted. Notably, the contours of the moisture distribution maps are very similar, showing that the lower moisture content (approximately 18% gravimetric water content) is located in an area 22 on the southern edge of the field, and the higher moisture content (up to 28% gravimetric water content) was measured in approximately the western area 24 of the field. As may be viewed when comparing FIGS. 17A and 17B, the moisture varied from approximately 17 to 25 wt %, and isolines with the same values of moisture are disposed approximately on the same places on both maps.

A second example is illustrated in FIGS. 17C and 17D, showing soil moisture distribution maps for a field referred to herein as Field 7. The map of FIG. 17C was constructed from gamma spectra acquired from a mobile PFTNA system and the map of FIG. 17D was constructed from TDR 350 soil moisture measurements taken on the same day as the PFTNA scan was conducted. In FIG. 17C, the map was based upon measurements taken at 25 locations evenly distributed across the field, represented by the black points on the map. These values were received by sorting and averaging by sites, shown in FIG. 14, the values of moisture calculated from the hydrogen peak areas obtained from the TNC midpoints spectra saved when surveying Field 7 with the PFTNA system. A large number of midpoints spectra (866 midpoints) were acquired and saved during a survey of this Field 7 that took approximately 8 hours to complete; however, as mentioned above, it is noted that a suitable number of points for moisture content determination, taken in approximately the same locations across Field 7, may be completed within approximately one hour, if other soil elements are not being measured. For comparison, a moisture distribution map of the same field is shown in FIG. 17D, which is based upon 350 moisture measurements taken across the Field 7 with a TDR-350, the locations of these measurements shown as the points in FIG. 17D. Again, the moisture distribution maps in FIGS. 17C and 17D are quite similar, showing a gravimetric water content varying between approximately 4 to 13% wt across the field.

### Temperature-Controlled Housing

In some embodiments of the PFTNA system, the system includes a temperature-controlled housing for housing the plurality of gamma detector assemblies and maintaining the plurality of gamma detector assemblies at a set temperature. For example, the gamma detector assemblies may comprise a plurality of Nal crystals coupled to a photomultiplier tube (PMT). The Applicant has discovered that the plurality of gamma detector assemblies, comprising at least the sodium iodine crystals operatively coupled to the PMTs, are susceptible to recording gamma ray spectra wherein the peaks in the spectra that are characteristic of different elements in the soil, may have centroids of the characteristic peaks shifted by several degrees when scanning operations are conducted in ambient temperatures that are much higher or lower than 20° C. This observed spectral shift is believed to introduce errors into the determination of peak areas and the resulting elemental soil analysis or soil moisture content as described above. The Applicant concludes that the observed spectral shifts are due to a change in detector gain with changes in temperature. As each detector assembly is slightly different and requires calibration, it would be difficult to obtain a calibration coefficient to account for these changes in detector gain based on changes in temperature.

Because such scanning operations may be conducted in a variety of climates and weather conditions, the Applicant provides a temperature-controlled housing for housing the plurality of gamma detector assemblies, to maintain the gamma detector assemblies at a stable temperature, so as to eliminate the impact of ambient temperature fluctuations on the detector gain.

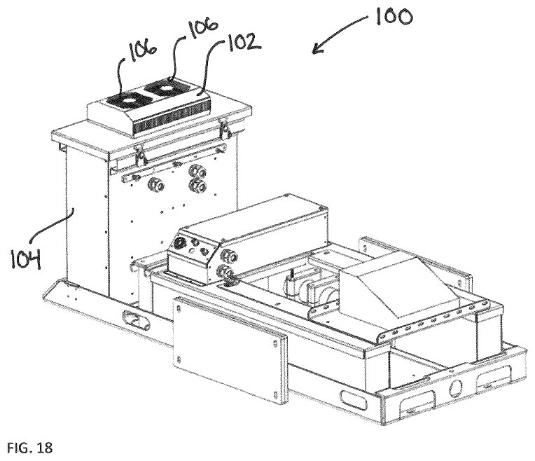

In some embodiments as shown in FIG. 18, a mobile PFTNA system 100, which may be mounted to a cart (not shown), comprises an array of detector assemblies (not shown) housed within a temperature-controlled housing 104. The temperature-controlled housing 104 is provided with a heating and cooling unit 102, which circulates heated or cooled air into the housing 104 via fans 106. The heating and cooling unit 102 may also be provided with a temperature sensor, such as a thermocouple or other sensors as would be known to a person skilled in the art. The temperature sensor may be in electronic communication with an electronic controller of the PFTNA system. The electronic controller monitors the temperature readings provided by the temperature sensor and controls the heating and cooling unit 102 so as to maintain the temperature within the housing 104 at a pre-determined temperature, within a tight temperature range. For example, not intended to be limiting, the pre-determined temperature of the housing 104 may be set at 20° C. If it is desired to maintain a peak stability of +/−1% (in other words, for example, a hydrogen peak having a centroid at 2.22 MeV would be expected to be positioned at 2.22 MeV+/−0.02 MeV), then the temperature within the housing 104 would be maintained at 20° C.+/−0.5° C. On the other hand, if peak stability is required to be maintained at +/−0.5%, then the temperature within the housing would be maintained at 20° C.+/−0.25° C.

In some embodiments, the heating and cooling unit 102 may be configured to maintain the pre-determined temperature when ambient temperature conditions fluctuate in the range between −20° C. and 40° C. In some embodiments, the heating and cooling unit may be provided with only a heater, so that the housing is heated to maintain the pre-determined temperature of, for example, 20° C. in cold weather conditions. In other embodiments, the heating and cooling unit may be provided with only an air conditioning unit, so that the housing is cooled to maintain the pre-determined temperature in hot weather conditions. In some embodiments, the housing 104 may include a double-wall construction and may also include insulation to assist with maintaining the temperature inside the housing at the pre-determined temperature.

What is claimed is:

1. A system for measuring a content of an element in a surface layer of a soil of a geographic area, the system comprising:
a neutron source for irradiating the surface layer of the soil with neutrons at a location within the geographic area,
a detector assembly comprising a plurality of gamma detectors, the detector assembly configured to detect at least an inelastic neutron scattering (INS) gamma spectrum of the surface layer of the soil at the location within the geographic area, an instrument for measuring a moisture content of the surface layer of the soil at the location,
a processor in communication with the detector assembly, the processor configured to:
associate the detected INS gamma spectrum with the geographic coordinates of the location where the detected INS gamma spectrum was acquired; apply a moisture calibration coefficient to calculate an amount of the element obtained from a net peak area of a characteristic peak of the element in the INS gamma spectrum, the net peak area obtained by subtracting a background peak area of a characteristic peak of the element from a measured peak area of the characteristic peak of the element in the detected INS gamma spectrum, the moisture calibration coefficient calculated to account for the moderation of fast neutrons by a quantity of hydrogen atoms present in the irradiated surface layer of the soil at the location under analysis, the quantity of hydrogen atoms present in the irradiated surface layer of the soil approximated by the moisture content of the surface layer of the soil as measured by the instrument, generate a concentration of the element in the surface layer of the soil at the location from the amount of the element obtained from the net peak area of the characteristic peak of the element in the INS gamma spectrum.

2. The system of claim 1, wherein the moisture calibration coefficient is calculated from simulated gamma spectra obtained from a simulated soil model, the simulated soil model comprising a plurality of simulated soil samples, each simulated soil sample of the plurality of soil samples containing a composition of elements including at least hydrogen, oxygen, carbon and silicon, the composition of elements in each simulated soil sample varying from the composition of elements in the other simulated soil samples of the plurality of simulated soil samples.

3. The system of claim 2, wherein the moisture calibration coefficient is calculated from a calibration data set, the moisture calibration coefficient normalized by multiplying the moisture calibration coefficient calculated from the calibration data set by a ratio of the value of the moisture calibration coefficient at zero moisture content obtained from the calibration data set divided by the value of the moisture calibration coefficient calculated from the simulated gamma spectra at zero moisture content, the calibration data set comprising a plurality of gamma spectra acquired from a plurality of calibration blocks, each calibration block of the plurality of calibration blocks comprising a known quantity of at least carbon and silicon.

4. The system of claim 3, wherein each calibration block of the plurality of calibration blocks contains less than 5% moisture.

5. The system of claim 1, wherein the detector assembly is configured to simultaneously detect the INS gamma spectrum and a thermal neutron capture (TNC) gamma spectrum of the surface layer of the soil, and wherein the instrument for measuring the moisture content of the surface layer of the soil comprises the detector assembly.

6. The system of claim 1, wherein the element is selected from a group comprising: carbon, silicon, oxygen, iron, aluminum.

7. The system of claim 5, wherein the system further comprises the neutron source, the detector assembly and the processor mounted to a mobile cart; and wherein the system is configured to measure the content of the element in the surface layer of the soil in each location of a plurality of locations across a geographic area with the processor configured to associate each detected INS and TNC gamma spectra of a plurality of detected INS and TNC gamma spectra with the geographic coordinates of each location where the detected INS and TNC gamma spectra was acquired; and wherein the processor is configured to generate the concentration of the element in the surface layer of the soil for each location of the plurality of locations.

8. The system of claim 7, wherein the system further comprises a global positioning system (GPS) and wherein the processor is configured to obtain the geographic coordinates of each location of the plurality of locations from the GPS.

9. The system of claim 7, wherein the processor is further configured to generate a map of the geographic area, the map indicating the concentration of the element in the surface layer of the soil for each location of the plurality of locations across the geographic area.

10. The system of claim 7, wherein the processor is further configured to calculate an average measured peak area of the characteristic peak of the element for a midway point located midway between two adjacent locations of the plurality of locations, the average measured peak area of the characteristic peak of the element calculated from two or more acquired INS and TNC gamma spectra obtained at each location of the two adjacent locations and between the two adjacent locations.

11. The system of claim 10, wherein the processor is further configured to generate a map of the geographic area, the map indicating the concentration of the element in the surface layer of the soil across the geographic area, the concentration of the element in the surface layer of the soil obtained from calculating the concentration of the element at each midway point based upon the average measured peak area associated with each midway point between two adjacent locations of the plurality of locations.

12. The system of claim 1 wherein the detector assembly is enclosed in a temperature-controlled housing, the temperature-controlled housing comprising a temperature sensor for detecting a temperature of the detector assembly, the temperature sensor in communication with a temperature controller, the temperature controller for receiving signals from the temperature sensor and actuating a heating unit to heat an interior of the housing when the temperature sensor detects a temperature of the detector assembly is less than a target temperature.

13. A system for measuring a concentration of at least one element in a surface layer of a soil in a geographic area, the system comprising:
a detector assembly comprising a plurality of gamma detectors, each gamma detector of the plurality of gamma detectors comprising a sodium iodine crystal coupled to a photomultiplier tube, each gamma detector of the plurality of gamma detectors configured to detect at least one gamma ray spectrum of each location of a plurality of locations across the geographic area,
the detector assembly enclosed in a temperature-controlled housing, the temperature-controlled housing comprising a temperature sensor for detecting a temperature of the detector assembly, the temperature sensor in communication with a temperature controller, the temperature controller for receiving signals from the temperature sensor and actuating a heating unit, the heating unit actuated to heat an interior of the housing when the temperature sensor detects a temperature of the detector assembly is less than a target temperature,
a processor in communication with the plurality of gamma detectors, the processor configured to:
associate a gamma ray spectrum with a geographic coordinates of each location of the plurality of locations where the gamma ray spectrum was acquired, and
calculate a concentration of the at least one element at each location of the plurality of locations within the geographic area based on a net peak area of a characteristic peak of the at least one element obtained from the acquired gamma spectrum.

14. The system of claim 13, wherein the temperature controller is further configured to actuate a cooling unit to cool an interior of the housing when the temperature sensor detects a temperature of the detector assembly is greater than a target temperature.

15. The system of claim 14, wherein the target temperature is 20° C.

16. The system of claim 14, wherein the system is configured to maintain the target temperature within a range of greater than or less than 0.5° C. of the target temperature to maintain a peak stability of greater than or less than 1% of the centroid of the characteristic peak of the at least one element.

17. The system of claim 14, wherein the system is configured to maintain the target temperature within a range of greater than or less than 0.25° C. of the target temperature to maintain a peak stability of greater than or less than 0.5% of the centroid of the characteristic peak of the at least one element.
